SEQUENCE LISTING (2 sequences)

SEQ ID NO: 1 - protein, 303 aa, Homo sapiens [sequence omitted]

SEQ ID NO: 2 - protein, 304 aa, Mus musculus
MKGKEEKEGGARLGTGGGGSPDKSPSAQELKEQGNRLFVGRKYPEAAACYGRAITRNPLVAVYYTNRALCYLKMQQPEQALADCRRALELDGQSVKAHFFLGQCQLEMESYDEAIANLQRAYSLAKEQRLNFGDDIPSALRIAKKKRWNSIEERRIHQESELHSYLTRLIAAERERELEECQRNHEGHEDDGHIRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

(19) United States
(12) Patent Application Publication
(10) Pub. No.: US 2023/0374087 A1
(43) Pub. Date: Nov. 23, 2023

(54) METHOD TO IMPROVE PROTEIN QUALITY CONTROL

(71) Applicant: The Johns Hopkins University, Baltimore, MD (US)

(72) Inventors: David A. Kass, Baltimore, MD (US); Mark J. Ranek, Baltimore, MD (US)

(21) Appl. No.: 18/027,671

(22) PCT Filed: Sep. 23, 2021
(86) PCT No.: PCT/US2021/051743

§ 371 Date: Mar. 22, 2023

Related U.S. Application Data
(60) Provisional application No. 63/082,137, filed on Sep. 23, 2020.

(51) Int. Cl. C07K 14/47; C12N 15/86; A61P 3/00

(52) U.S. Cl. CPC C07K 14/47; C12N 15/86; A61P 3/00

(57) ABSTRACT

The disclosure is directed to methods for inhibiting toxic protein accumulation in cells by modulating the phosphorylation of carboxy terminus of heat shock cognate 70 interacting protein (CHIP), as well as a method for treating a disease caused by proteotoxicity in cells by increasing or inducing the activity of CHIP in a subject.

### STATEMENT REGARDING FEDERALLY-SPONSORED RESEARCH

This invention was made with government support under grant numbers HL 135827 and HL119012 awarded by the National Institutes of Health. The government has certain rights in the invention.

### BACKGROUND

The ubiquitin proteasome system (UPS) regulates post-transcriptional protein stability by identifying and then either repairing or disposing of damaged/misfolded proteins (1). The degradation process employs a three-enzyme cascade (the E1 ubiquitin activating enzyme, E2 ubiquitin-conjugating enzymes, and E3 ubiquitin ligases) that ultimately conjugate multiple molecules of ubiquitin, an 8 kDa protein, to selective lysine residues on a target protein. The poly-ubiquitin chain is recognized by the 26S proteasome, and the ubiquitinated substrate is then degraded and recycled back into amino acids (1). In addition to having a constitutive role in protein homeostasis, the UPS is actively engaged in stress conditions where misfolded/damaged proteins accumulate, aggregate, and can cause/exacerbate disease (2). Such proteotoxicity contributes to a variety of heart diseases, including myocardial ischemia/reperfusion/infarction (3) and amyloidosis, and neurological disorders such as Alzheimer's disease and Parkinson's disease (4).

There remains a need for methods and compositions that improve and maintain protein quality control (PQC) in cells.

### BRIEF SUMMARY

The disclosure provides method of inhibiting toxic protein accumulation in a cell, which method comprises contacting a cell with a nucleic acid sequence encoding a carboxy terminus of heat shock cognate 70 interacting protein (CHIP), wherein the CHIP protein comprises at least one phosphomimetic amino acid substitution at position 19 of SEQ ID NO: 1 or at position 20 of SEQ ID NO: 2, whereby the nucleic acid sequence is expressed in the cell and toxic protein accumulation is inhibited.

Also provided is a method of inhibiting toxic protein accumulation in a cell, which method comprises increasing or inducing in the cell phosphorylation of a serine residue at (i) position 19 of a human carboxy terminus of heat shock cognate 70 interacting protein (CHIP) comprising the amino acid sequence of SEQ ID NO. 1 or at (ii) position 20 of a mouse CHIP protein comprising the amino acid sequence of SEQ ID NO: 2.

The disclosure also provides a method of treating a disease caused by proteotoxicity in a cell, which comprises increasing or inducing the activity of carboxy terminus of heat shock cognate 70 interacting protein (CHIP) in a subject in need thereof.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

FIG. 1A includes images and a graph illustrating that isolated myocytes subjected to stimulated ischemia (SI) show increased accumulation of ubiquitinated proteins (UP) that is amplified if phosphodiesterase 5A (PDE5A) is also expressed to block protein kinase G (PKG) activation (n=6/group). FIG. 1B is a graph showing that PDE5A overexpression results in reduced proteasome activity at both baseline and after SI (n=6/group). FIG. 1C includes images and a graph illustrating that SI increases CHIP protein expression after SI, which is blocked by PDE5Aoe. FIG. 1D is a graph showing that CHIP mRNA expression is unaltered between groups. FIG. 1E is an image of an immunoblot confirming diminished CHIP protein levels under basal and SI conditions by incubation with siRNA to CHIP. Control is scrambled siRNA (Scr) (see FIG. 8B). FIG. 1F is a graph illustrating the LDH/MTT ratio in NRCMs exposed to control or SI conditions, +/− siRNA to CHIP (100 pM) or Scr, and with or without a PDE5A inhibitor, sildenafil (SIL, 1 μM) (n=6/group). ** p<0.01; ***p<0.001; ****p<0.0001 by Tukey post-hoc testing following 1W-ANOVA.

FIG. 2A is an image of immunoprecipitation of Myc-tagged CHIP with PKG1a. Lanes: 1—input, 2—negative control, 3—IP signal after three bead washes. FIG. 2B is a schematic illustrating that mass spectroscopy detects increased Chip-S20 (mouse sequence) phosphorylation with acute PKG activation. The lower schematic shows the sequence and fragmentation matching the spectra in the top panel. The M2S spectra has red annotations for the assigned ion fragments, with three fragment ions localizing phosphorylation at S20 (b8 and y11 indicate it occurs on the N-terminal sequence of LGTGGGGS, b7 indicates the sequence LGTGGGG does not contain the site). FIG. 2C is an alignment of CHIP peptide sequences from different species. The beginning of the N-terminus shows a high level of conservation, including three serines (S19, S23, S25 for human; S20, S24, S26 for mouse). A lysine at K30 (human, K31 mouse) is critical for CHIP-chaperone binding and is highly conserved. FIG. 2D is a graph showing MS detection of serine phosphorylation of a recombinant human CHIP peptide fragment containing residues 13-30 (Uniprot Q9UNE7). Only S20 phosphorylation is detected with WT CHIP. If an S20A mutation is expressed, some reduced phosphorylation is observed at S24 and S26. FIG. 2E is an image of an immunoblot assay of recombinant PKG1a/CHIP, which shows a pS20 signal for full length WT but not for the S20A mutant. FIG. 2F is an image showing that phospho S20 antibody detects CHIP phosphorylation at S20 in rat myocytes exposed to cGMP or PDE5A inhibition (n=3-4/group); both were blocked by concomitant PKG inhibition with DT3 (1 μM).

FIG. 3A is an image of a representative Western blot of ubiquitinated proteins and summary densitometry for NRCMs infected with adenovirus expressing WT, S20E (SE), or S20A (SA) CHIP, and exposed to normal or SI conditions for 48 hours. * p<0.05; *** p<0.001; **** p<0.0001; Tukey test, 1W-ANOVA. FIG. 3B is a graph illustrating the cytotoxicity (LDH/MTT assays) due to SI is reduced by (SE) and increased by SA CHIP expression as compared to WT. PKG activation by PDE5A inhibitor sildenafil (SIL) reverses cytotoxicity in WT, and to a lesser extent with both SE or SA expression. Overall ANOVA p<10⁻⁶. Symbols for Tukey post hoc multiple comparisons: * p<10⁻⁶ versus SI without SIL, †p<10⁻⁶ vs SE or SA+SI, #p<10⁻⁶ vs SE+SI; and ‡p<10⁻⁶ for SA+SI+SIL versus WT+SI+SIL and SE+SI+SIL. FIG. 3C includes graphs showing that proteasome activity in resting NRCMs is augmented by PKG activation with SIL in both control cells (top panel) and those subjected to SI (bottom panel). Results of 2W-ANOVA for each condition are provided. There was a significant increase in activity with SIL, regardless of the CHIP form expressed, and no interaction between these variables. Data are n=6/group for each of the panels.

FIG. 4A includes images of M-mode echocardiograms which show more left ventricular (LV) dilation and dysfunction post-myocardial infarction (MT) in mPDE5/oe versus littermate controls 1-week following MI. FIG. 4B includes graphs of summary data for left ventricular ejection fraction (n=7−8/group) and fibrosis (n=3−8/group) for the same study. LV function, and morphology (n=7−8/group) (heart weight and lung weight normalized to tibia length (HW/TL, LW/TL, respectively). P values: * p<0.05; ** p<0.005; *** p<0.0005; **** p<0.0001 by multiple comparisons test following 2-way ANOVA or KW test. FIG. 4C is an image illustrating that ubiquitinated protein increases after MI in the peri-infarct zone are augmented in mPDE5/oe hearts (FIG. 11A) (n=6/group). FIG. 4D includes an image of a representative Western blot and a graph of summary data (n=6/group) for total CHIP expression in the same four conditions. FIG. 4E includes images of myocardial sections stained for CHIP (green), cardiac troponin T (cTnT, red), and nuclei (DAPI, blue). There is less myocyte CHIP expression in mPDE5/oe myocardium post MI. FIG. 4F includes images from an example immunoblot for pS20 and total CHIP from the peri-infarct zone myocardium prior to and post-MI. FIG. 4G includes graphs summarizing the results of the immunoblot in FIG. 4F (n=6/group). FIG. 4H is a graph showing that myocardial PKG activity directly correlates with levels of p/t CHIP. Data are from MI study with rest and post MI data pooled in the two groups.

FIG. 5A is structural model of the binding region between Hsc70 lid motif and the TPR region in CHIP. The location of S20 in reference to opposing lysine residues K597 and K601 in Hsc70 is shown. FIG. 5B includes images of in vitro expression of Myc-tagged WT, SE, or SA CHIP which show markedly reduced Hsc70-CHIP co-precipitation when the phospho-null SA form was expressed (summary data in FIG. 14A) (n=4/group). FIG. 5C includes images of immunoprecipitation gels and summary data for Hsc70 and CHIP interaction. CHIPHsc70 co-precipitation increased following MI in vivo but not if PDE5A was overexpressed (summary data in FIG. 14B) (n=4/group). FIG. 5D includes images and a graph showing that PKG enhances CHIP post-translational half-life. NRCMs were infected with AdV-Flag or AdV-Flag-PDE5 and cultured in media containing cycloheximide (CHX, 100 μM) to block protein synthesis and H₂O₂ (50 μM) to induce oxidative stress. An additional group received PDE5A inhibition (1 μM SIL) to stimulate PKG (n=4−5/group; ** p<0.01; **** p<0.0001 by analysis of covariance. FIG. 5E includes images and a graph of results from the same experimental protocol as in FIG. 5D but using NRCMs over-expressing S20A or S20E CHIP mutants. P-value for ANCOVA. FIG. 5F is a graph illustrating results from the same experimental protocol as in FIGS. 5D and 5E, but comparing WT and K31A CHIP mutant, the latter displaying a shorter half-life. P-value for ANCOVA. FIG. 5G is a graph of results from a Hsp70/Hsp40 dependent protein refolding ATPase assay showing capacity of CHIP to reduce activity in a dose dependent manner in wild type (WT), S20A, and S20E CHIP mutants. In contrast, K31A CHIP expression prevented this negative modulation (n=9/group). * p<0.006; ** p<0.001 vs no CHIP added (blue bar).

FIG. 6A is a graph illustrating that CHIP_S20E (n=10 heterozygous; n=7 homozygous) mice have reduced mortality following myocardial infarction due to total coronary occlusion. FIG. 6B includes an image of whole heart histology which reveals a larger infarct territory with replacement fibrosis in WT versus CHIPS20E hearts (see graph, right panel). FIG. 6C includes images of M-mode echocardiograms which show improved wall motion and less heart dilation in CHIPS20E versus WT controls (FIGS. 6B, 6C: heterozygous n=3/homozygous n=2). FIG. 6D includes graphs of summary data for LV function and volumes, which illustrate substantial protection against MI in CHIPS20E. FIG. 6E includes images showing that protein ubiquitination (UB) is reduced post MI in CHIPS20E mice. (n=6/group, *** p<0.001; **** p<10−6.). FIG. 6F includes images of immunoblots for total CHIP protein expression at baseline and post-MI in the peri-infarct zone from WT and CHIPS20E mice (summary data in FIG. 16). FIG. 6G is an image from a filter dot assay for insoluble proteins probed for ubiquitin or CHIP. UB proteins are reduced with CHIP levels increased in CHIPS20E myocardium (FIGS. 6E, 6F, 6G: heterozygous n=3, homozygous n=3).

FIGS. 7A and 7B are graphs showing that in vitro overexpression of PDE5A suppresses PKG activity (FIG. 7A) and CHIP abundance (FIG. 7B) during simulated ischemia (SI). PKG activity in isolated myocytes infected with adenovirus expressing Flag protein, or Flag-PDE5A, and then subjected to normal conditions or 48 hours of simulated ischemia. PDE5A overexpression prevented PKG activation from SI. 1W-ANOVA; Tukey post-hoc comparisons test. *** p<0.001; **** p<0.0001.

FIG. 8A is a graph illustrating cytotoxicity assessed by LDH/MTT ratio in isolated neonatal myocytes overexpressing WT, or S24E or S24A mutant CHIP. Data shown at baseline, after SI, and with SI co-treated with the PDE5A inhibitor sildenafil (SIL). Unlike the data shown in FIG. 3B related to S20 modification, mutating S24 had no significant impact on the cytotoxic response to SI or SI+SIL. ANOVA followed by Sidak's post hoc tests: * p<10⁻⁶ vs corresponding SI without SIL; †p<10⁻⁶ vs corresponding baseline (no SI or SIL), ‡p=0.02 vs WT SI. FIG. 8B is a graph illustrating results of the same experiment as in FIG. 8A, but comparing WT versus S20E/S24E and S20A/S24A combination mutants. These results are very similar to those with S20 mutations alone. ANOVA followed by Sidak's posthoc testing: †p<10⁻⁶ vs SE+SI and SA+SI, * p<10⁻⁶ vs corresponding SI without SIL, ‡p<10⁻⁶ vs WT+SI+SIL, and SE+SI+SIL.

FIG. 9 is a graph showing that PDE5 overexpression in mice increased PDE5 activity and suppressed PKG activity following myocardial infarction. The graph shows results of PDE5A and PKG enzyme activity assays in peri-infarct region at baseline and following myocardial infarction in mice with myocyte-specific PDE5Aoe versus littermate controls. 2WANOVA, Tukey Post hoc testing: **** p<10⁻⁶, * p<0.05.

FIG. 10 is a graph showing that PDE5 overexpression in mice exacerbates heart failure phenotype following myocardial infarction. Heart weight and lung weight were both normalized to tibia length (HW/TL; LW/TL respectively). Mice with myocyte PDE5Aoe showed greater increases in both after myocardial infarction (MI). 2WANOVA, post hoc tests: * p<0.05; *** p<0.001.

FIGS. 11A and 11B demonstrate the accumulation of ubiquitinated proteins following myocardial infarction (MI) is augmented in hearts with PKG activity blunted due to PDE5Aoe. FIG. 11A is a graph summarizing data for the immunoblots shown in FIG. 4C. 1way-ANOVA; Tukey multiple comparisons test **** p<10⁻⁶. FIG. 11B includes confocal microscopy images of immunohistochemistry of myocardium with antibody staining for ubiquitin (green), troponin T (red), and nuclei (DAPI, blue). Myocardium from mPDE5Aoe post-MI hearts shows increased myocyte ubiquitinated protein accumulation compared to that in WT mice from the same stimulus. Baseline levels are very low in both. In WT, the ubiquitinated protein accumulates in the interstitium, whereas the mPDE5Aoe is in myocytes only, and the accumulation is localized to these cells. This supports the linkage between PKG activity and ubiquitination.

FIG. 12A is a graph showing proteasome activity in the peri-infarct region from WT and mPDE5Aoe hearts in normal and post-MI conditions. PDE5A overexpression reduced intrinsic proteasome activity similarly with or without the infarct. 1W-ANOVA, Tukey post-hoc comparisons test p-values shown. FIG. 12B is a graph showing that CHIP gene expression (normalized to GAPDH expression) is similar regardless of the condition or genotype. FIG. 12C is a graph showing mass spectroscopic detection of phospho-serine 20 on CHIP protein from myocardial extracts in control and post-MI peri-infarct regions in PDE5Aoe and littermate control mice (n=5−7/group).

FIGS. 13A and 13B illustrate sex disparities in cardiac functional parameters and CHIP levels (gene and protein) following MI. FIG. 13A includes graphs showing that female mice subjected to MI display significantly less myocardial dysfunction and chamber dilation as compared to males. P-values are shown for unpaired t-test between groups. FIG. 13B is a graph showing that the degree of increased CHIP expression is greater in female myocardium post MI than male myocardium (2W-ANOVA, p values for Sex and Sham vs MI are provided; post hoc comparisons: ** p<0.01; ***p<0.001; ****p<0.0001. FIG. 13C is a graph showing that CHIP gene expression (Stub1/Gapdh) is greater in females, and this disparity remains after MI. * p=0.048 versus male MI.

FIG. 14A is a graph summarizing data for the gel shown in FIG. 5, which shows CHIP-Hsc70 co-immunoprecipitation with CHIP-WT, CHIP-S20E, and CHIP-S20A forms. There is a rise in co-precipitation with CHIP-S20E after SI, whereas CHIP-S20A has minimal co-precipitation at baseline or after SI. ANOVA followed by Tukey multiple comparisons test. *** p=0.0005 vs SE without SI; * p=0.048 vs WT+SI, †p<5×10⁻⁶ vs all other groups. FIG. 14B is a graph summarizing data for the immunoblot shown in FIG. 5B. * p<0.05 by Dunn's test, following Kruskal Wallis analysis.

FIG. 15 is a schematic illustrating the generation of a S20E knock-in (KI) mouse using CRISPR/Cas9 for gene editing. The left panel shows guide RNA to target CHIP S20/S24 region, and the replacement RNA that includes a silent mutation of the PAM sequence to further enhance homology-directed repair. The right panel shows PCR sequences used to generate primers for genotyping.

FIG. 16 is a graph summarizing the data of the Western blot analyses from FIG. 6F for CHIP and total protein in CHIPS20E/S20E (homozygous) mice and littermate WT controls following sham surgery or MI surgeries. There was a significant increase in CHIP expression in WT mice but not in mice expressing CHIP-SE (data from 1W-ANOVA).

FIG. 17 is a graph showing gene expression of Stub1/Gapdh in CHIPS20E/S20E mice and littermate WT controls, with either sham surgery or coronary ligation to induce myocardial infarction (MI). There was a significant reduction of expression in mice expressing CHIP-SE, and a slight but significant rise overall following MI (data from 2WANOVA).

FIG. 18 includes confocal microscopy images showing increased transthyretin (TTR) positive aggregates in Chip SA NRCMs but not in Chip SE NRCMs.

FIG. 19 includes images of a western blot showing that TTR accumulated in the Chip WT human induced pluripotent stem cell derived cardiomyocytes (hiPSC-CMs), particularly with Chip SA expression, but was cleared by Chip SE expression. Stimulating PKG was able to reduce TTR accumulation in Chip WT cells but not in Chip SA cells.

FIG. 20 includes images of a western blot showing that Lamp2A knock-down exacerbated the accumulation of TTR in the Chip WT and SA cells and removed the protective effect of Chip SE.

FIG. 21 includes images of a western blot showing that TTR accumulated in the Chip WT cardiac fibroblasts, particularly with Chip SA expression, but was cleared by Chip SE expression in a dose-dependent manner.

### DETAILED DESCRIPTION

The present disclosure is predicated, at least in part, on the discovery that protein turnover mediated by carboxy terminus of heat shock cognate 70 interacting protein (CHIP) is markedly post-translationally enhanced by direct protein kinase G (PKG) phosphorylation of the serine residue at position 19 (S19) and position 20 (S20) of the human and mouse CHIP proteins, respectively. Phosphorylation of this serine residue increases CHIP binding affinity to Hsc70, CHIP protein half-life, and consequent clearance of stress-induced ubiquitinated-insoluble proteins. As disclosed herein, PKG-mediated phosphorylation of CHIP, or a nucleic acid sequence expressing a CHIP phospho-mimetic, reduces ischemic proteo- and cytotoxicity, whereas a phospho-silenced CHIP amplifies both. In addition, the present disclosure demonstrates that reducing PKG activity in vivo reduces CHIP phosphorylation, exacerbating proteotoxicity and heart dysfunction after ischemic injury. Thus, the present disclosure demonstrates that PKG activation provides post-translational enhancement of protein quality control via CHIP.

### Definitions

To facilitate an understanding of the present technology, a number of terms and phrases are defined below. Additional definitions are set forth throughout the detailed description.

The terms “protein homeostasis” and “proteostasis” are used interchangeably herein and refer to the sum total of protein synthesis (i.e., translation), post-translational processing and transport, folding, assembly and disassembly into macromolecular complexes, stability, and clearance. Protein homeostasis encompasses an entire landscape that takes into account all aspects of the processes that underlie a cell's stable or unstable protein complements. Thus, transcriptional, translational, and post-translational regulation all form a part of this landscape as do the dynamic processes of folding, unfolding, post-translational modifications, trafficking, and degradation. If any one of these processes is perturbed by internal or external stimuli, a cell must respond to either regulate that specific process or compensate by regulating some other function. If a protein contains a mutation that predisposes it to incorrect folding or aggregation, or some other process is disturbed such that protein misfolding/aggregation occurs, proteotoxicity may result. The term “proteotoxicity,” as used herein, refers to any impairment of cellular function as a result of protein misfolding or aggregation. As used herein, the term “protein quality control (PQC)” refers to the collection of innate mechanisms cells possess to sense protein unfolding and/or misfolding and restore normal protein conformations. The mechanisms underlying PQC are complex and include constitutive and inducible chaperones, but also compartment-specific mechanisms.

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

An amino acid “replacement” or “substitution” refers to the replacement of one amino acid at a given position or residue by another amino acid at the same position or residue within a polypeptide sequence. A “phospho-mimetic amino acid substitution” refers to an amino acid substitution that mimics a phosphorylated protein, thereby activating (or deactivating) the protein. Phospho-mimetic amino acid substitutions may occur at residues that are commonly phosphorylated in vivo, such as serine, tyrosine, and threonine residues.

As used herein, the term “preventing” refers to prophylactic steps taken to reduce the likelihood of a subject (e.g., an at-risk subject) from contracting or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition within the scope and meaning herein.

As used herein, the terms “treatment,” “treating,” and the like, refer to obtaining a desired pharmacologic and/or physiologic effect against a particular disease, disorder, or condition. Preferably, the effect is therapeutic, i.e., the effect partially or completely cures the disease and/or adverse symptom attributable to the disease.

### CHIP and Protein Homeostasis

Proteotoxic insults, such as the accumulation of misfolded proteins, can lead to cell death and dysfunction, and give rise to severe phenotypes in tissues such as the brain and heart. Indeed, it is well known in the art that protein misfolding can lead to cardiovascular disease. In addition, the improper folding or maintenance of protein structure can lead to a protein's improper degradation, such as occurs in cystic fibrosis (cystic fibrosis transmembrane conductance regulator protein) and Gaucher disease (β-glucosidase) (Qu et al., J Bioenerg Biomembr., 29: 483-490 (1997); and Demuro et al., J Biol Chem., 280: 17294-17300 (2005)). Many neurodegenerative diseases (e.g., Alzheimer's disease and Parkinson's disease) are the result of the accumulation of multiple, misfolded species which form toxic polypeptides and aggregates. Protein quality control (PQC) pathways have evolved to maintain protein fidelity in the face of intrinsic and extrinsic insults and prevent misfolded proteins from accumulating. Such pathways include, for example, chaperones, which include the heat shock proteins (HSP), the ubiquitin proteasome system (UPS), the autophagy-lysosome pathway, and the ER-associated degradation (ERAD) stress response (see, e.g., McLendon, P. M., Robbins, J., Circ Res., 116(11): 1863-1882 (2015)). Together, these PQC pathways normally ensure that the balance of protein synthesis to protein degradation (i.e., protein homeostasis) is maintained, functioning independently, and in concert with one another to recognize and eliminate proteotoxic threats (Willis, M. S., Patterson, C., N Engl J Med., 368: 455-464 (2013)).

The ubiquitin proteasome system (UPS) regulates post-transcriptional protein stability by identifying and then either repairing or disposing of damaged/misfolded proteins (1). The degradation process employs a three-enzyme cascade (the E1 ubiquitin activating enzyme, E2 ubiquitin-conjugating enzymes, and E3 ubiquitin ligases) that ultimately conjugate multiple molecules of ubiquitin, an 8 kDa protein, to selective lysine residues on a target protein. The poly-ubiquitin chain is recognized by the 26S proteasome, and the ubiquitinated substrate is then degraded and recycled back into amino acids (1). In addition to having a constitutive role in protein homeostasis, the UPS is actively engaged in stress conditions where misfolded/damaged proteins accumulate, aggregate, and can cause/exacerbate disease (2). Such proteotoxicity contributes to a variety of heart diseases, including myocardial ischemia/reperfusion/infarction (3) and amyloidosis, and neurological disorders such as Alzheimer's and Parkinson's disease (4).

The present disclosure is based on the phosphorylation status of a serine residue of the carboxyl terminus of the Hsc70-interacting protein or CHIP. The CHIP protein, encoded by the Stub1 gene, is an important member of the UPS system, functioning as both an E3-ligase and co-chaperone, and facilitating protein degradation (5). CHIP is ubiquitously expressed in mammals, with the highest protein levels found in hypermetabolic tissues such as cardiac and skeletal muscle. The linear structure of CHIP is comprised of triple tandem tetratricopeptide repeat (TPR)-domains in the N-terminal, a central coiled coil domain, and a C-terminal U-box domain that confers its ligase activity (6). The TPR domain and the adjacent charged region of ˜200 amino acids form the binding site to heat shock proteins (HSP), primarily Hsc70, Hsp70, and Hsp90. HSP-CHIP interactions can modify substrate targeting and can suppress chaperone-refolding. Nucleotide and amino acid sequences of human and mouse CHIP are publicly available from the GenBank database under Accession Nos. AF129085 and AF129086, respectively. The amino acid sequence of human CHIP is represented by SEQ ID NO: 1, and the amino acid sequence of mouse CHIP is represented by SEQ ID NO: 2.

The disclosure provides methods of inhibiting toxic protein accumulation in a cell. The phrase “inhibiting toxic protein accumulation,” as used herein, refers to the ability of a substance or method to interfere with the accumulation of misfolded and/or damaged proteins in a cell. The degree of inhibition may be partially complete (e.g., 10% or more, 25% or more, 50% or more, or 75% or more), substantially complete (e.g., 85% or more, 90% or more, or 95% or more), or fully complete (e.g., 98% or more, or 99% or more). In some embodiments, the disclosure provides a method of inhibiting toxic protein accumulation in a cell which comprises contacting a cell with a nucleic acid sequence encoding a carboxy terminus of heat shock cognate 70 interacting protein (CHIP), wherein the CHIP protein comprises a serine to glutamic acid substitution at position 19 (S19E) of SEQ ID NO: 1 or at position 20 (S20E) of SEQ ID NO: 2. It will be appreciated that the serine to glutamic acid substitution at positions 19 and 20 of SEQ ID NO: 1 and SEQ ID NO: 2, respectively, mimics a phosphorylated protein (i.e., is “phospho-mimetic”). As discussed above, nucleic acid sequences encoding human and mouse CHIP are publicly available under Accession Nos. AF129085 and AF129086, respectively. In certain embodiments, the nucleic acid sequence is in the form of a vector. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 4th edition, Cold Spring Harbor Press, Cold Spring Harbor, N.Y. (2012), and Ausubel et al., Current Protocols in Molecular Biology, Greene Publishing Associates and John Wiley & Sons, New York, N.Y. (1994)).

In some embodiments, viral vectors may be used to deliver the CHIP-encoding nucleic acid sequence to cells. Viral vectors used in the art to deliver and express exogenous genes in mammalian cells include, for example, retrovirus (see, e.g., Cavazzana-Calvo et al., Science, 288 (5466): 669-672 (2000)), lentivirus (see, e.g., Cartier et al., Science, 326: 818-823 (2009)), adeno-associated virus (AAV) (see, e.g., Mease et al., Journal of Rheumatology, 27(4): 692-703 (2010)), herpes simplex virus (HSV) (see, e.g., Goins et al., Gene Ther., 16(4): 558-569 (2009)), vaccinia virus (see, e.g., Mayrhofer et al., J. Virol., 83(10): 5192-5203 (2009)), and adenovirus (see, e.g., Lasaro and Ertl, Molecular Therapy, 17(8): 1333-1339 (2009)). Any suitable viral vector may be used in the context of the present disclosure, a variety of which are available from commercial sources. Other genetically modified virus and virus-like particles may be employed as a vector, such as those described in, e.g., International Patent Application Publication WO 2019/104279 and Roldão et al., “Viruses and Virus-Like Particles in Biotechnology: Fundamentals and Applications,” Comprehensive Biotechnology, 633-656; doi:10.1016/B978-0-12-809633-8.09046-4 (2017).

In addition to the nucleic acid encoding CHIP, the vector desirably comprises expression control sequences, such as promoters, enhancers, polyadenylation signals, transcription terminators, internal ribosome entry sites (IRES), and the like, that provide for the expression of the nucleic sequence in a host cell. Exemplary expression control sequences are known in the art and described in, for example, Goeddel, Gene Expression Technology: Methods in Enzymology, Vol. 185, Academic Press, San Diego, Calif. (1990).

In some applications, it may be appropriate to deliver the CHIP-encoding nucleic acid sequence in the form of a synthetic mRNA construct. Synthetic mRNA-based therapeutics are being developed for delivery into various cell types and for applications as diverse as cancer vaccination, cell reprogramming, and protein replacement therapies (von Niessen et al., Mol Ther., 27(4): 824-836 (2019)). mRNA constructs are often advantageous over DNA-based vectors (e.g., plasmids or virus vectors), mainly due to their ease of manipulation and safety in clinical applications (Leonhardt et al., Nanomedicine, 10: 679-88 (2013).

A nucleic acid sequence encoding CHIP, or a vector comprising same, may be introduced into a host cell that is capable of expressing the polypeptide encoded thereby, such as a prokaryotic cell or a or eukaryotic cell. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5a, DH10, MC1061 (ATCC No. 53338), and CC102). Suitable eukaryotic cells are known in the art and include, for example, yeast cells, insect cells, and mammalian cells. Examples of suitable yeast cells include those from the genera Hansenula, Kluyveromyces, Pichia, Rhinosporidium, Saccharomyces, and Schizosaccharomyces. Suitable insect cells include Sf-9 and HIS cells (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants also are suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for culture, amplification, screening, and purification of such cells are well known in the art (see, e.g., Ausubel et al., eds., Short Protocols in Molecular Biology, 5th ed., John Wiley & Sons, Inc., Hoboken, N.J. (2002)). Preferably, the mammalian cell is a human cell.

A nucleic acid sequence encoding CHIP, or a vector comprising same, may be introduced into a cell by any suitable method, including, for example, by transfection, transformation, or transduction. The terms “transfection,” “transformation,” and “transduction” are used interchangeably herein and refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); strontium phosphate DNA co-precipitation (Brash et al., Mol. Cell. Biol., 7: 2031-2034 (1987); and nanoparticle-based gene delivery (e.g., magnetic nanoparticle-based gene delivery (Dobson, J., Gene Ther, 13 (4): 283-7 (2006)).

In some embodiments, the disclosure further provides a method of inhibiting toxic protein accumulation in a cell (e.g., any cell type described herein), which comprises increasing or inducing in the cell phosphorylation of a serine residue at (i) position 19 of a human carboxy terminus of heat shock cognate 70 interacting protein (CHIP) comprising the amino acid sequence of SEQ ID NO: 1 or at (ii) position 20 of a mouse CHIP protein comprising the amino acid sequence of SEQ ID NO: 2. “Increasing” phosphorylation of a specific CHIP protein residue refers to the ability of a substance or method to augment or elevate the rate or level at which CHIP phosphorylation occurs as compared to the rate or level of phosphorylation in the absence of the substance or method. “Inducing” phosphorylation of a specific CHIP protein residue refers to the ability of a substance or method to activate or stimulate CHIP phosphorylation from a state of inactivity in the absence of the substance or method.

Increasing or inducing phosphorylation of human and mouse CHIP may be accomplished by, for example, increasing or inducing the activity of kinase(s) that are directly or indirectly involved in CHIP phosphorylation. Alternatively, CHIP phosphorylation may be increased or induced by inhibiting compounds or processes that negatively regulate CHIP phosphorylation. In some embodiments, increasing or inducing phosphorylation of Ser19 of human CHIP or Ser20 of mouse CHIP comprises increasing or inducing the activity of protein kinase G (PKG) in the cell. Protein kinase G (PKG) (also known in the art as “cGMP-dependent protein kinase”) is the primary effector for the second messenger cyclic guanosine monophosphate (cGMP) generated by nitric oxide or natriuretic peptide stimulation. PKG phosphorylates a number of biologically important targets and is implicated in the regulation of smooth muscle relaxation, platelet function, sperm metabolism, cell division, and nucleic acid synthesis. PKG activation was previously reported to reduce protein aggregation and associated proteotoxicity from accumulated misfolded proteins in both myocytes and heart muscle by stimulating the proteasome (22, 23).

In some embodiments, increasing or inducing the activity of PKG in a cell comprises contacting a cell with an agent that stimulates PKG activity. A variety of agents and compounds that stimulate PKG activity are known in the art and can be used in the disclosed methods. Such agents include, but are not limited to, nitric oxide donors, organic nitrates, soluble guanylate cyclase stimulators, PDE5 inhibitors that enhance NO-dependent cGMP synthesis, natriuretic peptides (NP), neprilysin inhibitors, and PDE9 inhibitors that increase NP-dependent cGMP stimulation. Exemplary nitric oxide donor drugs include diazeniumdiolates, S-nitrosothiols, NO-NSAID compounds, zeolites, and are further described in, e.g., Miller, M. R., and Megson, I. L., Br J Pharmacol., 151(3): 305-321 (2007)). Exemplary organic nitrate drugs include nitroglycerin, isosorbide dinitrate, erythrityl tetranitrate, and entaerythritol tetranitrate (see, e.g., B. Soman, G. Vijayaraghavan, E-Journal of Cardiology Practice, 15(2) (2017)). Exemplary soluble guanylate cyclase stimulators include the amino dicarboxylic acid cinaciguat, the synthetic benzylindazole compound YC-1, and pyrazolopyridine derivatives BAY 41-2272 and BAY 41-8543 (see, e.g., Stasch et al., Circulation, 123(20): 2263-2273 (2011)). Exemplary PDE5 inhibitors include sildenafil, avanafil, lodenafil, and mirodenafil (see, e.g., Andersson, K-E., Br. J Pharmacol., 175(13): 2554-2565 (2018)). Exemplary natriuretic peptides include nesiritide, ularitide, and carperitide (see, e.g., Meems, L. M. G., Burnett, J. C., Jr., JACC Basic Transl Sci., 1(7): 557-567 (2016)). An exemplary neprilysin inhibitor is sacubitril/valsartan (see, e.g., Riddell, E., Vader, J. M., Curr Heart Fail Rep., 14(2): 134-145 (2017)). Exemplary PDE9 inhibitors include pyrimidinone derivatives, 3r, BAY 73-6691, PF-04447943, and BI-409306 (see, e.g., Shao et al., J. Med. Chem., 57(24): 10304-10313 (2014); and Wu et al., Acta Pharmaceutica Sinica B, 8(4): 615-628 (2018)).

In other embodiments, the method comprises altering an endogenous CHIP gene in the cell, wherein the altered CHIP gene encodes a protein comprising (a) a substitution of the serine residue at position 19 of SEQ ID NO: 1 for glutamic acid (S19E) or aspartic acid (S19D) or (b) a substitution of the serine residue at position 20 of SEQ ID NO: 2 for glutamic acid residue (S20E) or aspartic acid (S20D). “Altering” a gene or DNA sequence refers to modifying at least one physical feature of a wild-type DNA sequence of interest. DNA alterations include, for example, single or double strand DNA breaks, deletion or insertion of one or more nucleotides, and other modifications that affect the structural integrity or nucleotide sequence of the DNA sequence. An endogenous CHIP gene may be altered using any suitable method for introducing targeted, sequence-specific changes to a nucleic acid sequence. For example, gene editing methods may be used to alter the endogenous CHIP gene. The terms “gene editing” and “genome editing,” as used herein, refer to technologies that allow genetic material to be inserted, removed, or altered at a particular location in the genome. Gene editing technologies that may be used in the context of this disclosure include, but are not limited to, zinc-finger nucleases (ZFNs), transcription activator-like effector nucleases (TALENs), and CRISPR (clustered regularly interspaced short palindromic repeat DNA sequences)/Cas9. Gene editing technologies are further described in, e.g., Appasani, K. (ed.), Genome Editing and Engineering: From TALENs, ZFNs and CRISPRs to Molecular Surgery, 1st ed., Cambridge University Press (2018); and Hirakawa et al., Biosci Rep. 2020; 40(4):BSR20200127. doi:10.1042/BSR20200127.

The methods described herein may be performed in cells in vitro or in vivo. The term “in vivo” refers to a method that is conducted within living organisms in their normal, intact state, while an “in vitro” method is conducted using components of an organism that have been isolated from its usual biological context. When the methods are performed in cells in vitro, the cell may be any suitable prokaryotic or eukaryotic cell, such as those described herein. When the methods are performed in cells in vivo, the methods may be performed in an animal, such as a mammal, particularly a human. In some embodiments, the cell is in a human and the nucleic acid sequence encoding a substituted CHIP protein is present in a composition comprising a pharmaceutically acceptable carrier.

In some embodiments, the human suffers from cardiac dysfunction or disease. As used herein, “cardiovascular dysfunction,” “cardiovascular disease,” “CVD,” and “cardiovascular disorder” are terms used to classify numerous conditions affecting the heart, heart valves, and vasculature (e.g., veins and arteries) of the body and encompass diseases and conditions including, but not limited to arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, gestational hypertension, pre-eclampsia, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), myocardial ischemia-reperfusion injury, and cerebrovascular disease. In some embodiments, the cardiovascular dysfunction or disease is myocardial ischemia-reperfusion injury. Acute myocardial infarction (AMI) is a leading cause of morbidity and mortality in the world (Mozaffarian et al., Circulation, 131(4): e29-e322 (2015)). While reperfusion strategies are the current standard therapy for AMI, they may result in paradoxical cardiomyocyte dysfunction and worsen tissue damage, which is known as “reperfusion injury” (Yellon, D. M. and Hausenloy, D. J., The New England Journal of Medicine, 357(11): 1074-1135, (2007); and Verma et al., Circulation, 105 (20): 2332-2336 (2002)). Ischemic reperfusion injury (IRI) typically arises in patients presenting with an acute ST-segment elevation myocardial infarction (STEMI). In other embodiments, the cardiac dysfunction is the result of amyloid accumulation in the myocardium. For example, the cardiac dysfunction may be transthyretin cardiac amyloidosis (ATTR-CA). Transthyretin cardiac amyloidosis (ATTR-CA) is an infiltrative cardiomyopathy caused by extracellular deposition of insoluble transthyretin (TTR) amyloid fibrils in the myocardium (Ruberg F L and Berk J L, Circulation, 126: 1286-1300 (2012)). TTR is a plasma protein mainly synthesized in the liver, recognized as a transporter of thyroxine and retinol-binding protein. Unstable changes in two different types of TTR (wild type or variant) lead to misfolding, aggregation, and ultimately formation of amyloid fibrils. Cardiac amyloidosis (CA) has been identified as a cause of heart failure with preserved ejection fraction (HFpEF) among elderly persons, and its incidence has been constantly increasing because the population is aging. CA is progressive and life-threatening if left untreated, and thus, early diagnosis is critical (Yamamoto, H. and Yokochi, T., ESC Heart Fail., 6(6): 1128-1139 (2019)).

In other embodiments, the human may suffer from neurological dysfunction or disease. The terms “neurological dysfunction,” “neurological disease,” and “neurological disorder,” as used herein, refer to diseases or conditions of the central and peripheral nervous system, which includes the brain, spinal cord, cranial nerves, peripheral nerves, nerve roots, autonomic nervous system, neuromuscular junction, and muscles. Neurological diseases include, but are not limited to, epilepsy, Alzheimer's disease, Huntington's disease, Parkinson's disease, dementia (e.g., Alzheimer's disease-related dementias (ADRDs)), prion disease, amyotrophic lateral sclerosis (ALS), cerebrovascular diseases including stroke, migraine, and other headache disorders, multiple sclerosis, neuroinfections, brain tumors, traumatic disorders of the nervous system due to head trauma, and neurological disorders as a result of malnutrition. The disclosure also provides a method of treating a disease caused by proteotoxicity in a cell, which comprises increasing or inducing the activity of carboxy terminus of heat shock cognate 70 interacting protein (CHIP) in a subject in need thereof. The disease to be treated may be any suitable disease or disorder that arises as a result of proteotoxicity in cells (also referred to herein as “proteinopathies”), such as any of the cardiovascular or neurological diseases or disorders described herein. Other proteinopathies that may be treated include, but are not limited to, cystic fibrosis transmembrane regulator (CFTR) opathies, laminopathies, inflammasomeopathies, adiposopathy, telomeropathies, Fanconi anemia bloom syndrome (FANC-BLM) opathies, lysosomopathies, phosphatopathies, phosphotase and tensin homolog deleted on chromosome 10 (PTEN) opathies, caspase recruitment domain family member 11 (CARD1l or CARMA1)—B cell CLL/lymphoma 10 (BCL10)—MALT1 paracaspase (MALT1) (CBM) opathies, phosphodiesterase (PDE) opathies, BCID cargo adapter 2 (BICD2) opathies, Behcet's disease, axonopathy, dysferopathy, synaptopathy, clippers and myelin oligodendrocyte glycoprotein (MOG) opathy, RAS/mitogen-activated protein kinase (RAS) opathies, protein kinase R-like ER kinase (PERK) opathies, Bcl-2-associated athanogene (BAG) opathies (e.g., Bcl-2-associated athanogene-1 gene(BAG1) opathy or Bcl-2-associated athanogene-3 gene (BAG3) opathy), BRG1/BRM-associated factor (BAF) opathies/SWI/SNF-related intellectual disability disorder (SSRIDD), four and a half LIM domain 1 (FHL1) opathies, apolipoprotein E (APOE) opathies, tauopathies, Abetaopathies (also referred to as “amyloid beta-opathies”), Huntington's disease (e.g., Huntingtonoapthy), alpha-synucleinopathies (e.g., synucleinopathies), multiple sclerosis, transactive response DNA-binding protein 43 (TDP-43) opathies, fused-in-sarcoma (FUS) opathies, superoxide dismutase 1 (SOD1) opathies, amyotrophic lateral sclerosis (ALS), microtubuleopathies, heat shock protein (HSP) opathies, chaperoneopathies, Paget's disease, osteoprotegerinopathies, glycogen synthase kinase 3 beta (GSK3beta)opathies, oligodendrogliopathies, fronto-temporal lobar degeneration (FTLD), British and Danish dementias, cerebral autosomal dominant arteriopathy with subcortical infarcts and leukoencephalopathy (CADASIL), neurogenic locus notch homolog protein 3 (NOTCH3) opathies, prion disease, Creutzfeldt-Jakob disease, Pick's disease, traumatic brain injury (TBI), hypertrophic cardiomyopathy, cardiac arrhythmias, channelopathies, Noonan syndrome, sarcomeropathies, and myofibrillar myopathy.

As described above, in some embodiments, increasing or inducing the activity of CHIP comprises administering to the subject a composition comprising a nucleic acid sequence encoding a human CHIP protein comprising a serine to glutamic acid substitution at position 19 (S19E) of SEQ ID NO: 1 and a pharmaceutically acceptable carrier, whereby the nucleic acid sequence is expressed in a subject and the disease is treated. In other embodiments, increasing or inducing the activity of CHIP comprises increasing or inducing the activity of protein kinase G (PKG) in the cells of the subject. The activity of PKG may be increased or induced using any suitable substance or method, including contacting the cell with an agent that stimulates PKG activity, such as any agent described herein or known in the art.

When a cell is contacted, in vitro or in vivo, with a composition comprising a nucleic acid sequence encoding a human CHIP protein comprising a serine to glutamic acid substitution at position 19 (S19E) of SEQ ID NO: 1 and a pharmaceutically acceptable (e.g., physiologically acceptable) carrier, a variety of suitable formulations of the composition are possible. The choice of carrier will be determined, in part, by the particular use of the composition (e.g., administration to an animal) and the particular method used to administer the composition. For example, the composition may contain preservatives, such as, for example, methylparaben, propylparaben, sodium benzoate, and benzalkonium chloride. A mixture of two or more preservatives optionally may be used. In addition, buffering agents may be included in the composition. Suitable buffering agents include, for example, citric acid, sodium citrate, phosphoric acid, potassium phosphate, and various other acids and salts. A mixture of two or more buffering agents optionally may be used. Suitable compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The compositions can be presented in unit-dose or multi-dose sealed containers, such as ampules and vials, and can be stored in a freeze-dried (lyophilized) condition requiring only the addition of the sterile liquid carrier, for example, water, immediately prior to use. Methods for preparing compositions for pharmaceutical use are known to those skilled in the art and are described in more detail in, for example, Remington: The Science and Practice of Pharmacy, Lippincott Williams & Wilkins; 21st ed. (May 1, 2005).

One of ordinary skill in the art will appreciate that the nucleic acid sequence can be present in a composition with other therapeutic or biologically-active agents. For example, factors that control inflammation, such as ibuprofen or steroids, can be part of the composition to reduce swelling and inflammation associated with in vivo administration of the composition.

A composition comprising a CHIP-encoding nucleic acid sequence, or an agent that increases or induces the activity of protein kinase G (PKG), can be administered to a human using standard administration techniques and routes. Suitable administration routes include, but are not limited to, oral, intravenous, intraperitoneal, subcutaneous, subcutaneous, or intramuscular administration. The composition ideally is suitable for parenteral administration. The term “parenteral,” as used herein, includes intravenous, intramuscular, subcutaneous, rectal, vaginal, and intraperitoneal administration.

For in vivo applications, any suitable dose of the CHIP-encoding nucleic acid sequence, or the agent that increases/induces PKG activity, may be administered to a mammal (e.g., a human), so long as the nucleic acid sequence or agent is efficiently delivered to cells such that toxic protein accumulation therein is inhibited. To this end, the inventive method comprises administering a “therapeutically effective amount” of the CHIP-encoding nucleic acid sequence or the agent that increases/induces PKG activity. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the CHIP-encoding nucleic acid sequence or the agent that increases/induces PKG activity to elicit a desired response in the individual. For example, a therapeutically effective amount of the CHIP-encoding nucleic acid sequence or the agent that increases/induces PKG activity is an amount which reduces or completely blocks proteotoxicity in cells, such that a cardiac or neurological disease is eliminated.

Alternatively, the pharmacologic and/or physiologic effect may be prophylactic, i.e., the effect completely or partially prevents a disease or disorder caused by proteotoxicity. In this respect, the inventive method comprises administering a “prophylactically effective amount” of the CHIP-encoding nucleic acid sequence or the agent that increases/induces PKG activity. A “prophylactically effective amount” refers to an amount effective, at dosages and for periods of time necessary, to achieve a desired prophylactic result (e.g., prevention of a cardiac or neurological disease).

Therapeutic or prophylactic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the invention.

The disclosed method can be performed in combination with other therapeutic methods to achieve a desired biological effect in a human patient. For example, when the method is performed in a human suffering from cardiac dysfunction or disease, the disclosed method may include, or be performed in conjunction with, one or more treatments for cardiovascular disease. Suitable cardiovascular disease treatments that may be employed include, but are not limited, interventions such as angioplasty, bypass surgery, and pacemakers, or pharmaceuticals such as angiotensin converting enzyme (ACE) inhibitors, aspirin, beta blockers, calcium channel blockers, nitrates, and blood thinners. When the method is performed in a human suffering from neurological dysfunction or disease, a variety of therapies are available depending on the particular neurological dysfunction or disease. For example, a human suffering from Alzheimer's disease may be treated with Donepezil (ARICEPT®), rivastigmine (EXELON®), and galantamine (RAZADYNE®) to manage mild to moderate AD symptoms, while a human suffering from Parkinson's disease may be treated with levodopa combined with carbidopa, anticholinergic drugs, pramipexole, apomorphine, and/or ropinirole. Treatments for ALS include, but are not limited to, physical therapy, baclofen, diazepam, gabapentin, trihexyphenidyl, and amitriptyline. Surgical methods also may be utilized in certain types of neurological disorders. The foregoing treatments are not limiting, however, and the disclosure encompasses any treatments for cardiovascular or neurological diseases known in the art.

The following examples further illustrate the invention but, of course, should not be construed as in any way limiting its scope.

### EXAMPLES

The following methods and materials were used in the experiments set forth herein.

### Mouse Models

Studies used mice (C57BL6/J background) with myocyte-specific PDE5A overexpression under control of an α-MHC tetracycline-inducible promoter (24) and their littermate controls. A new global knock-mouse expressing CHIP S20E/S24E (CHIP SE) also was generated using CRISPR/Cas9 targeting/insertion methods (Transgenic Mouse Core Laboratory, Johns Hopkins University).

### Myocardial Infarction (MI)

Male and female mice (aged ˜3-4 months) were subjected to transmural myocardial infarction by mid left anterior descending artery ligation under open thoracotomy (25). Controls underwent open chest surgery and LAD isolation without ligation. Data obtained 1-week after infarction included 2D echocardiography (Vevo2100, 20-MHz; Visualsonics) in conscious mice to measure LV volume and ejection fraction, with analysis blinded to study group. The protocol was approved by the Johns Hopkins Animal Care and Use Committee.

### Cultured Cardiomyocyte Studies

Primary cultures of neonatal rat cardiomyocytes (NRCMs) were prepared as described (26). After overnight culture, subsets of NRCMs were infected with adenovirus (AdV) containing a FLAG-tagged PDE5, FLAG control, Myc-tagged CHIP WT or K31A (provided by Dr. Cam Patterson), S20E or S20A (Welgen Inc., Worcester, MA). Forty-eight hours after infection, cells were exposed to either simulated ischemia (SI) or normal conditions. SI involved incubating cells in DMEM containing deoxyglucose (20 mM), NaCl (125 mM), KCl (8 mM), KH2PO4 (1.2 mM), MgSO4 (1.25 mM), CaCl2) (1.2 mM), NaHCO3(6.25 mM), sodium lactate (5 mM), and HEPES (20 mM) with a pH of 6.6. Cells were then placed in an incubator chamber (Billups-Rothenberg) with 95% nitrogen, 4% carbon dioxide, and 1% oxygen. Normoxic controls were incubated in DMEM supplemented with 10% FBS in air composed of 95% carbon dioxide and 5% oxygen. RNA or protein was harvested after 48 hours of SI. Cells were also treated with sildenafil (1 μM) or vehicle, or small interference RNA (siRNA, 100 pM) specific for rat Stub1 (Qiagen, Cat. #: SI01532251) or scrambled control provided 24 hours after plating and transfected with Xfect (Takara) per manufacturer protocol.

### Protein Analysis

Tissue and cultured cardiomyocytes were lysed in 1× lysis buffer (Cell Signaling Technology), protein concentration was measured by bicinchoninic acid assay (Pierce), and extracts were subjected to SDS-PAGE using Novex Tris-Glycine Gels (4-20% gradient gel, Life Technologies) or Mini-protean TGX Gels (4-20% gradient gel, Bio-Rad), transferred to nitrocellulose membranes, and probed with various primary antibodies (CHIP (#2080S), Hsc70 (#8444S), p-CHIP S20 (Custom antibody, AbMart Inc., Shanghai, China; Project: 25011-1), Myc (#2276S), GAPDH (#2118S), and α-tubulin (#3878S) (Cell Signaling) and ubiquitin antibody (#SAB4503053) from Sigma), and fluorescence-labeled secondary antibodies (Licor, #926-32211, 926-32210, 926-68023, or 926-68022). Gels were imaged (Odyssey, Licor) and band intensity quantified (Odyssey Software 3.1).

### Proteasome Activity Assay

Proteasome peptidase activity assays were performed as described (22) with slight modification.

### LDH/MTT Cytotoxicity Assay

Lactate dehydrogenase (LDH) leakage and (3-[4,5-dimethylthiazol-2-yl]-2,5-diphenyltetrazolium bromide) (MTT) were assessed in cultured cardiomyocytes and media after SI from the same culture dish. LDH was assayed by commercial assay (Roche) following manufacturer's protocol, read in 96 well microplates on a Spectramax M5 (Molecular Devices) at 490 nm absorbance. For MTT, cells were washed in phosphate-buffered saline, MTT (Sigma-Aldrich) working solution added to the culture dish, incubated at 37° C., solubilized with acidic isopropanol, transferred to a 96 well plate, and read at 570 nm with background subtraction at 650 nm on a Spectramax M5.

### Cycloheximide (CHX) Chase Assay

CHX chase assays were performed as described (22). Neonatal rat ventricular myocytes (NRVMs) were incubated in serum-free DMEM containing 10 μM hydrogen peroxide to induce oxidative stress and 100 μM CHX (Sigma-Aldrich) to block further protein synthesis. Cells were harvested at consecutive time points after CHX and whole-cell lysates analyzed by immunoblot for CHIP or Myc (Myc-CHIP).

### Tissue Histology/Immunostaining

Myocardium was fixed in 4% paraformaldehyde, paraffin embedded, and sectioned into 4 μm slices, and stained with Masson's Trichrome. Fibrosis was quantified by color-based pixel count and intensity in parallel sections of the ischemic and remote areas (Aperio Image Scope). Transmural infarct size was estimated as the arc angle subtended by dense scar in a mid-MI cross section divided by 360° and expressed as percent. Immunostaining for ubiquitin (Sigma) and CHIP (Cell Signaling) was performed using AlexaFluor 488 conjugated secondary antibodies following the manufacturer's protocol. Counterstain was performed using DAPI (Invitrogen) and Troponin-T (Thermo Scientific) antibodies. Image acquisition was performed on a Leica confocal microscope (TCS SPE II) at 40× magnification.

### Quantitative Real-Time PCR

Total RNA was extracted with Trizol Reagent (Invitrogen) from myocardium or isolated myocytes per manufacturer's instructions, then reverse transcribed into cDNA using a High Capacity RNA-to-cDNA Kit (Applied Biosystems, Life Technologies). cDNA was subjected to PCR amplification using either TaqMan specific primers (Applied Biosystems) for CHIP (Stub1, mouse #Mm00490634_m1, rat #Rn01751757_g1) or gylceraldehyde-3-phosphate dehydrogenase (GAPDH, mouse #99999915_g1, rat #Rn01775763_g1) (Applied Biosystems). The threshold cycle (Ct) values determined by crossing point method, were normalized to the respective housekeeping gene GAPDH (Applied Biosystems) values for each run.

### PKG and PDE5A Activity Assay

In vitro PKG activity was measured by colorimetric assay (Cyclex), and PDE5A activity by fluorescence polarization assay (Molecular Probes) following manufacture protocols.

### Recombinant Proteins for In Vitro Assays

Human HSP40 (DnaJB1), human CHIP and CHIP point mutants in pET30a with a C-terminal His-tag and hHsc70 (HSPA8) wild type with an N-terminal His-tag in pCOLA-Duet1 were expressed in BL21(DE3) and purified on Ni-NTA (Qiagen). Hsc70 and CHIP proteins were induced and expressed for 22 hours at 18° C. Hsp40 protein was induced and expressed for 5 hours at 25° C. Required stock solutions of proteins were prepared by diluting high concentrated stocks in protein storage buffer into protein storage buffer, 20 mM HEPES (pH 7.4), 150 mM NaCl. Human, active PKG1α purified from Sf9 cells was purchased (SRP0371, Sigma).

### PKG and CHIP/CHIP-S20A In Vitro Kinase Assays

Aliquots of recombinant CHIP or PKG1α were diluted to either 12.5 μM or 0.25 μM in Kinase Dilution Buffer II (K23-09, SignalChem: 5 mM MOPS, pH 7.2, 2.5 mM β-glycerol-phosphate, 5 mM MgCl₂, 1 mM EGTA, 0.4 mM EDTA, 50 ng/μl BSA, 0.5 mM DTT). A 250 μM stock solution (5×) of ATP was made in Kinase Assay Buffer I (K01-09 SignalChem: 25 mM MOPS, pH 7.2, 12.5 mM β-glycerol-phosphate, 25 mM MgCl₂, 5 mM EGTA, 2 mM EDTA, 0.25 mM DTT). A 100 μM cGMP solution was purchased (#G47-09, SignalChem). The final buffer composition of complete reactions was 10.5 mM MOPS, 4 mM Beta-glycerol-phosphate, 8 mM MgCl₂, 1.6 mM EGTA, 0.64 mM EDTA, 0.08 mM DTT, 10 μM cGMP, 50 μM ATP. CHIP or PKG1α were used at final concentrations of 2.5 μM or 0.1 μM, respectively (25:1 molar ratio). Reactions were started with the addition of the 5× ATP solution, and incubated for 30° C. for 1 hour. Aliquots were either flash frozen for phospho-proteomic analysis or used for reducing SDS-PAGE/immunoblot analysis. Controls included no substrate (CHIP) or no kinase (PKG1a). Experimental triplicates were run on multiple days to confirm findings.

Mass spectrometry was performed at the UNC-CH Proteomics Center (Chapel Hill, NC, USA) as described (27). Samples were reduced, alkylated, and digested with trypsin overnight. After digestion, peptides were desalted using C18 spin columns and dried down via vacuum centrifugation. Each sample was analyzed in technical duplicate by LC/MS/MS using the Easy nLC 1200-QExactive HF (Thermo). Data were searched against a Uniprot E. coli database, appended with human CHIP-WT, CHIP-S19A, and PKG1α sequences, using Sequest within Proteome Discoverer (v2.1). Data were filtered using an FDR of 1% and peak areas were mean normalized.

### Isothermal Titration Calorimetry

Isothermal titration calorimetry was carried out using a TA Instruments NanoITC equilibrated at 25° C. and utilizing a stir speed of 600 rpm. Experiments were conducted in triplicate and data represents the average±one standard deviation. Titrations utilized 350 μM WT or S20E CHIP titrated into 50 M Hsp70 (385-641) as a series of twenty four 2 μL injections.

### Chaperone Refolding ATPase Activity Assay

Human HSP40 (DnaJB1), human CHIP and CHIP point mutants in pET30a with a C-terminal His-tag and hHsc70 (HSPA8) wild type with an N-terminal His-tag in pCOLA-Duet1 were expressed in BL21(DE3) and purified on Ni-NTA (Qiagen). Hsc70 and CHIP proteins were induced and expressed for 22 hours at 18° C. Hsp40 protein was induced and expressed for 5 hours at 25° C. Required stock solutions of proteins were prepared by diluting high concentrated stocks in protein storage buffer into protein storage buffer, 20 mM HEPES (pH7.4), 150 mM NaCl. Hsc70 and Hsp40 were used at a concentration of 0.5 μM and 0.25 μM, respectively, with a gradient of CHIP protein concentrations (0.5-6 μM). Hsp40-stimulated ATPase activity of Hsc70 was measured using fluorescent polarization with the TRANSCREENER® ADP2 FP Assay (BellBrook Labs, 3010-1K) on a Clariostar plate reader (BMG) in 384 well black, non-binding plates (Greiner, 784900). Optimized instrument settings were used: Excitation wavelength. 590 nm; Emission wavelength: 675 nm; Required value: 5%; Target mP: 20; Gain A: 1824; Gain B: 1854; Focal Height: 12.4 mm. Values of μM ADP formed in the reactions were interpolated from an ATP/ADP standard curve run in parallel.

### Statistical Analysis

Data are presented by the individual experimental results along with mean±SEM. Group comparisons were performed by 1- or 2-way ANOVA. Multiple comparisons testing was performed by Holm-Sidak or Tukey's test using Prism 8 software. Data that were not normally distributed were assessed by non-parametric analysis (Kruskal Wallis) test and Dunn's test for multiple comparisons. Sample sizes and individual statistical results for all analyses are provided in the figures or figure descriptions.

### Example 1

This example demonstrates that PKG activity regulates protein quality control (PQC) in a CHIP-dependent manner.

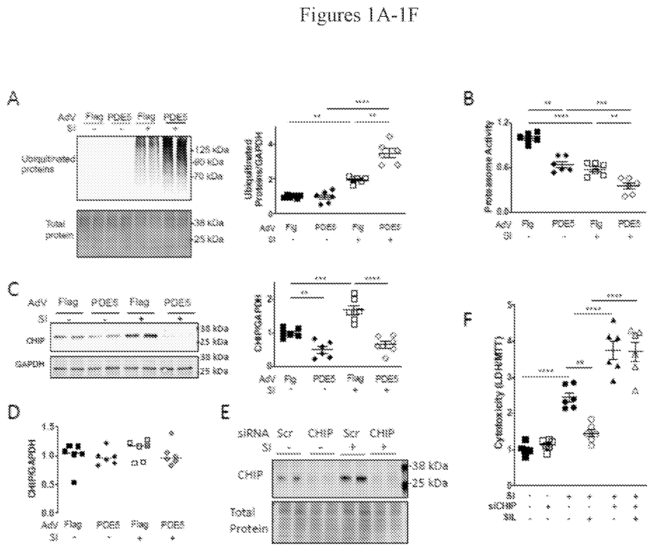

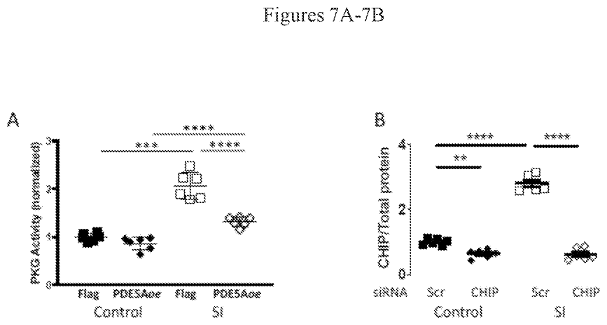

To test the intrinsic role of protein kinase G activation on PQC, loss of function studies were performed in cultured neonatal rat left ventricular cardiomyocytes transduced with an adenovirus expressing either epitope (Flag) tagged cGMP-selective phosphodiesterase 5 (PDE5Aoe) to degrade cGMP and block PKG activation, or Flag alone (FIG. 7A). Exposure to 48 hours of simulated ischemia (SI; deoxyglucose+acidosis+hypoxia) increased ubiquitinated proteins (UP) that further rose in cells with PDE5Aoe (FIG. 1A). PDE5oe also reduced proteasome activity in control myocytes and in those exposed to SI (FIG. 1B), the latter paralleling increased UP. Given the important role of CHIP in myocardial protein turnover, steady-state levels of CHIP were measured under these same conditions and it was found that, while SI increased CHIP protein quantity, CHIP protein levels were reduced by PDE5Aoe. PDE5oe also reduced CHIP protein levels in controls, but this was amplified after SI (interaction p<0.05, FIG. 1C). This change was post-translationally driven, as CHIP gene expression was unchanged (FIG. 1D).

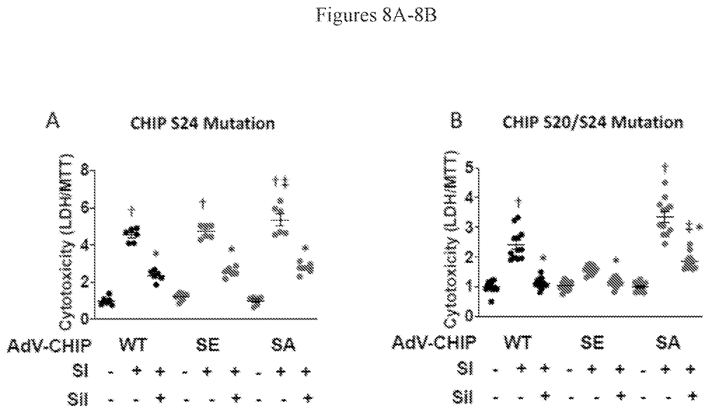

To test the importance of PKG-CHIP modulation on PQC after SI, myocytes were transfected with CHIP-siRNA or a scrambled control-siRNA construct, and then cotreated with or without the PDE5A inhibitor sildenafil (SIL, 1p M), which stimulates PKG by suppressing cGMP hydrolysis (FIG. 1E, FIG. 8B). SIL reduced SI-induced cytotoxicity in cells expressing CHIP, however this protective effect was lost in cells lacking CHIP (FIG. 1F). Moreover, silencing CHIP worsened cytotoxicity to SI.

Thus, the results of this example demonstrate that PKG assists intrinsic protein degradation by enhancing proteasome activity and by augmenting CHIP protein expression, reducing cytotoxicity with SI.

### Example 2

This example demonstrates that PKG directly phosphorylates mouse CHIP at Ser20 in cells.

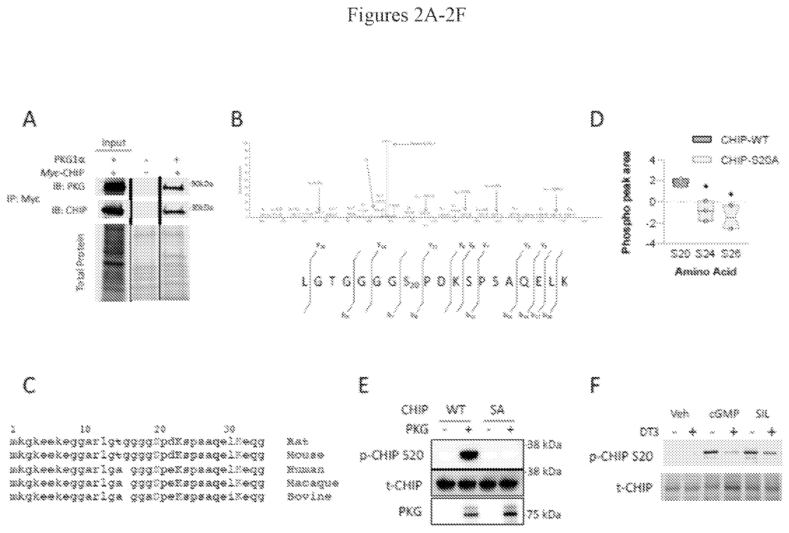

To determine if the involvement of PKG and CHIP occurs via direct protein-protein interactions, myc-tagged CHIP was expressed as bait in HEK293 cells and it was found that PKG is a co-immunoprecipitant (FIG. 2A). In a prior mass spectrometry (MS)-derived database of proteins in adult rat myocytes subjected to 15 min PKG activation (28), CHIP was found to be modified at S20 (S20 in rat and mouse, S19 human, FIG. 2B), a highly conserved residue in the TPR domain (FIG. 2C). Human CHIP peptide fragments containing amino acid residues 13-30 were incubated with cGMP/PKG and assayed by MS. Phosphorylation was observed at S20 with wildtype (WT) CHIP but not a phospho-silenced CHIP-S20A mutation. Phosphorylation was also observed at neighboring S24 and S26, but only when a S20A CHIP mutant was used (FIG. 2D). This indicates the primary target for PKG on CHIP is at S20. A monoclonal phospho-antibody generated against the S20 site in CHIP was tested in vitro with recombinant PKG1α and CHIP, verifying a consistent phospho-S20-CHIP signal. Use of a CHIP-S20A mutant CHIP confirmed specificity (FIG. 2E). CHIP pS20 increased in cardiomyocytes in which PKG was stimulated by addition of cGMP or by PDE5 inhibition with SIL. Importantly, the increase in CHIP pS20 was prevented by co-blockade of PKG activation by the peptide DT3 (1 μM) (FIG. 2F).

Thus, the results of this example demonstrate that PKG interacts with and directly phosphorylates CHIP at S20.

### Example 3

This example demonstrates that genetic alteration of CHIP S20 alters ischemic proteotoxicity in myocytes in vitro.

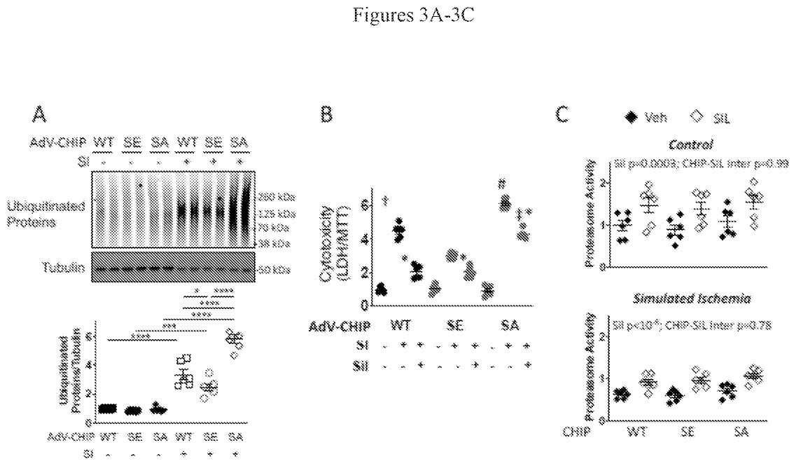

To directly probe the impact of S20 phosphorylation on proteotoxicity, adenovirus expression vectors encoding CHIP-WT, or CHIP-S20E, or CHIP-S20A were generated. Myocytes were infected to express one of these forms, and then subjected to SI for 48 hours. While each CHIP variant showed an increase in ubiquitinated proteins over non-stress controls, this was significantly amplified by CHIP-S20A and attenuated by CHIP-S20E expression, as compared to CHIP-WT (FIG. 3A). These differences were paralleled by disparities in SI-induced cytotoxicity (LDH/MTT ratio, FIG. 3B). Cells expressing CHIP-S20E mimicked the cytoprotection observed in CHIP-WT-expressing myocytes co-incubated with the SIL to stimulate PKG. In contrast, cells expressing CHIP-S20A displayed greater cytotoxicity and blunted the protective effects from SIL. Mutating the neighboring serine (S24 mouse, S23 human) had negligible impact on cytotoxicity, and combining S20 and S24 mutations yielded results similar to those with S20 modification alone (FIGS. 8A-8B).

Thus, the results of this example demonstrate that S20 is the primary effector of the cellular phenotype. The finding that SIL lowered cytotoxicity regardless of which CHIP protein was expressed, in both control and SI conditions (FIG. 3C), is consistent with PKG stimulation of proteasome activity (22).

### Example 4

This example demonstrates that PKG activity increases CHIP phosphorylation and protein expression and counters proteotoxicity after myocardial infarction in vivo.

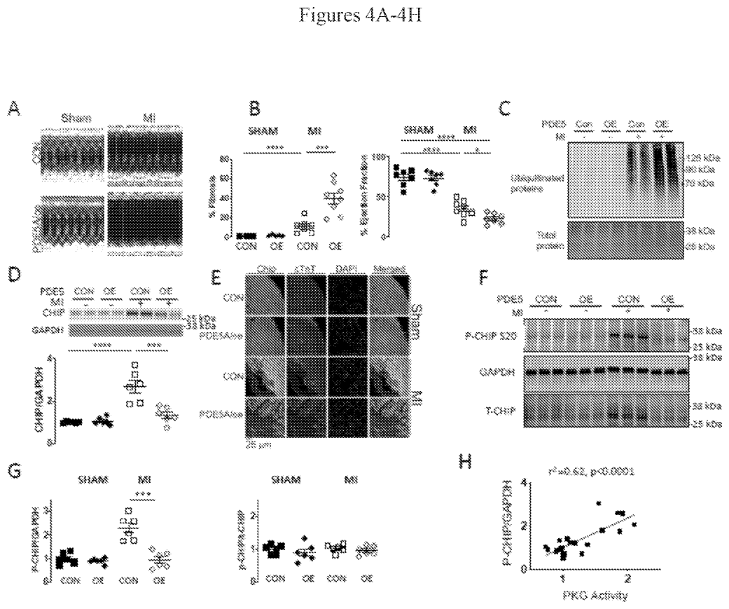

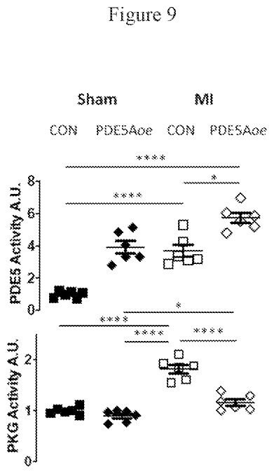

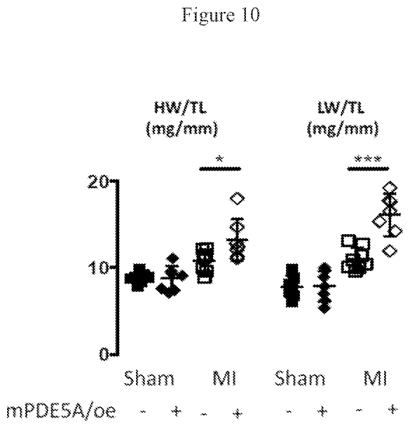

To test if intrinsic PKG activation regulates CHIP and protein quality control in vivo, mice overexpressing cardiomyocyte-restricted tetracycline-inducible phosphodiesterase 5 (mPDE5A/oe) were subjected to permanent coronary artery ligation to induce cardiac infarction. Without doxycycline co-treatment, myocyte PDE5A activity increases 3-4 fold over normal, preventing PKG activation in myocardium subjected to stress (24). There was no difference in PKG activity between sham operated mPDE5Aoe or controls, but it nearly doubled after MI in controls while remaining at basal levels in mPDE5A/oe hearts (FIG. 9). Preventing PKG activation after MI in mPDE5A/oe mice worsened subsequent heart function, increased pathological fibrosis (FIGS. 4A, 4B), and elevated both cardiac and lung weight (FIG. 10).

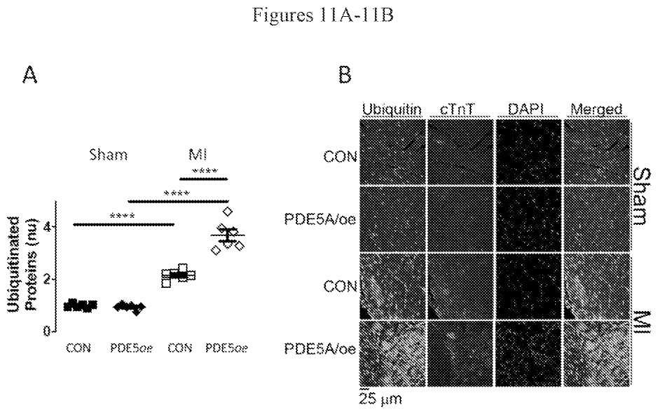

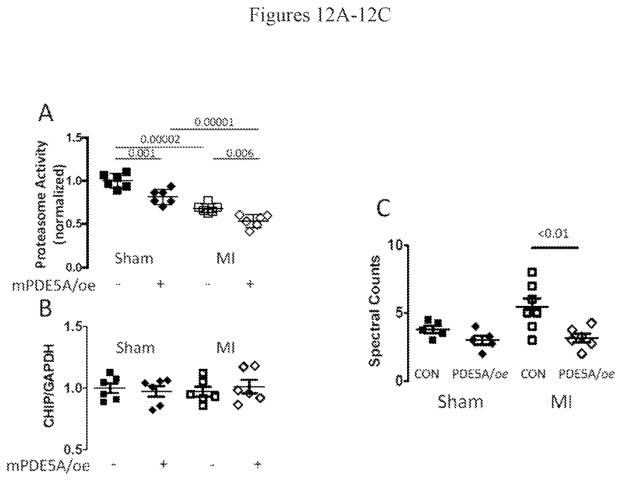

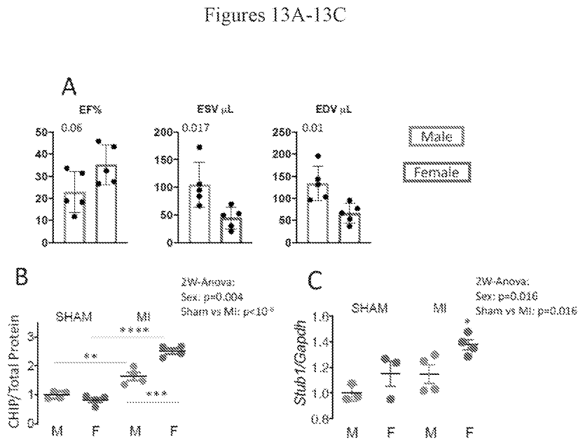

Global protein ubiquitination increased in the peri-infarct region after MI, and this was exacerbated in mPDE5A/oe mice (FIG. 4C, FIG. 11A). Confocal fluorescent microscopy showed that following MI, ubiquitinated proteins increased primarily in the interstitium in WT controls. However, in mPDE5A/oe hearts, the increase in ubiquitinated proteins appeared greatest in myocytes (identified by Troponin T co-localization; FIG. 11B), which is consistent with the myocyte-selective decline in PKG activity in mPDE5A/oe hearts. Increased myocyte abundance of PDE5A also correlated with reduced myocardial proteasome activity with or without MI (FIG. 12A). Although CHIP protein quantity increased after MI in control left ventricle (LV), this was not the case in the mPDE5A/oe mice (FIG. 4D). Differences in CHIP quantity between WT and mPDE5A/oe mice were also specific to cardiomyocytes, as shown by immunohistochemistry (FIG. 4E). As CHIP mRNA expression was again unaltered (FIG. 12B), increased protein was likely due to a post-translational modification. This is supported by increased pS20-CHIP in WT but not mPDE5A/oe hearts after MI, as detected by immunoblot (FIG. 4F) and MS (FIG. 12C). The decrease in both pS20 and total CHIP occurred in parallel, leaving the pS20/total CHIP ratio unaltered (FIG. 4G). This differs from the dissociation of pS20 from total CHIP with acute PKG modulation (FIG. 2F), suggesting the two become linked with chronic PKG suppression. pS20-CHIP levels also directly correlated with PKG activity measured in the same myocardium (FIG. 4H). Female mice subjected to the identical MI stress displayed considerably less dysfunction and chamber dilation as seen in males. Interestingly, enhanced female-dependent protective effects against MI were associated with greater CHIP gene and protein levels (FIGS. 13A-13B).

### Example 5

This example demonstrates that CHIP S20 phosphorylation augments and stabilizes CHIP-HSC70 binding and posttranslational protein stability.

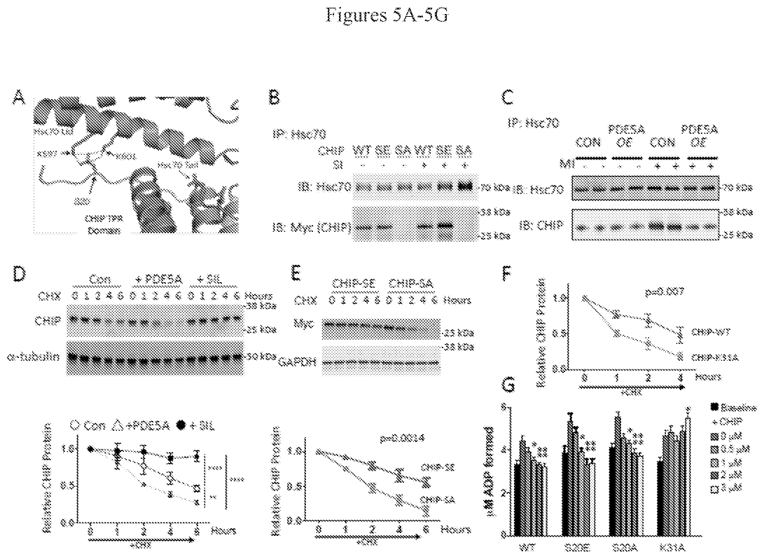

While CHIP has chaperone-independent activity, its binding to Hsc (Hsp70) is considered central to its function in promoting protein degradation (7, 8). S20 is located within the TPR domain of CHIP and coordinates with Hsc70 Lid-domain and the lysine residues (K597, K601) involved in protein-protein interactions (FIG. 5A). The thermodynamic impact of S20 phosphorylation on CHIP-Hsp70 binding was tested by isothermal titration calorimetry of CHIP-WT and CHIP-S20E. CHIP-S20E increased Hsp70 affinity by ˜0.5 μM (59%), coupled with a 5.6 kJ mol−1 more negative enthalpy (DH; −26.4±3.3 to −32.1±0.9, p<0.05) and a smaller change in entropy (DS, 25.5±8.3 to 9.8±3.7 J/Kmol, p<0.05) as compared to CHIP-WT. The DH and DS are consistent with a model based on the published structure of the CHIP-TPR/Hsp70-lid-tail complex (29) predicting that the S20E mutation (or S20 phosphorylation) would enhance the electrostatic interaction between CHIP and the Hsp70 lid domain, with an additional hydrogen bond potentially restricting motion of the lid with respect to CHIP-TPR, decreasing entropy for the CHIP/Hsp70 complex.

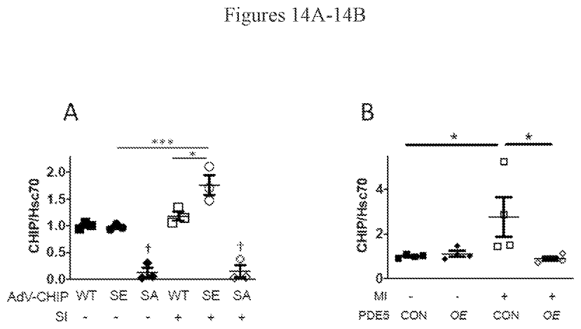

To test whether thermodynamic stabilization of CHIP-HSC70 due to S20 phosphorylation enhances protein binding, lysate from myocytes with or without exposure to SI were immunoprecipitated using Hsc70 as bait. Co-precipitation of CHIP and Hsc70 was confirmed in cells expressing CHIP-WT or CHIP-S20E, but co-precipitation was absent if CHIP-S20A was expressed (FIG. 5B, FIG. 14A). Reduced CHIP-Hsc70 interaction was also observed in vivo following MI in hearts with PKG activation suppressed by mPDE5A/oe, whereas this was enhanced in WT hearts (FIG. 5C, FIG. 14B).

The preceding results suggested that phosphorylation of CHIP at S20 enhances its protein half-life. To test this, myocytes were treated with cycloheximide (100 μM) to block protein synthesis and then exposed to hydrogen peroxide (50 μM) as a stress stimulus. Cells expressing only a Myc vector had a CHIP protein half-life of ˜6 hours (FIG. 5D), concordant with prior data (30). The half-life shortened to ˜2 hours if PKG activity was blocked by PDE5oe, but prolonged if PKG was stimulated by SIL. Using a similar protocol, it was found that CHIP-S20A protein half-life was significantly shorter than CHIP-S20E (FIG. 5E).

Mutation of CHIP Lys 31 (K30 in humans) to Ala is known to abolish CHIP chaperone interactions, and with it, the inhibitory effect of CHIP on chaperone ATPase activity required for their protein refolding function (8). As the impact of this mutation on CHIP half-life was not reported, it was examined here. The K30A mutation was found to indeed shorten half-life significantly to levels observed with S20A mutants (FIG. 5F). There was, however, a striking difference between the two mutations with respect to their inhibition of Hsp70 ATPase activity. As expected, HSP70 activity was dose-dependently inhibited by CHIP-WT, an effect that was lost with CHIP-K31A. However, both CHIP-S20E and S20A displayed similar efficacies as CHIP-WT, the latter despite its disruption of HSC-70 interaction (FIG. 5G).

The results of this example demonstrate that the S20A mutant destabilizes the CHIP and Hsc (Hsp) 70 interaction but does not prevent it, and chaperone activity is still modulated. However, loss of S20 phosphorylation favors CHIP protein degradation, and the reverse increased protein half-life and functionality.

### Example 6

This example demonstrates that knock-in mice expressing CHIP-S20E are protected against MI-proteotoxicity.

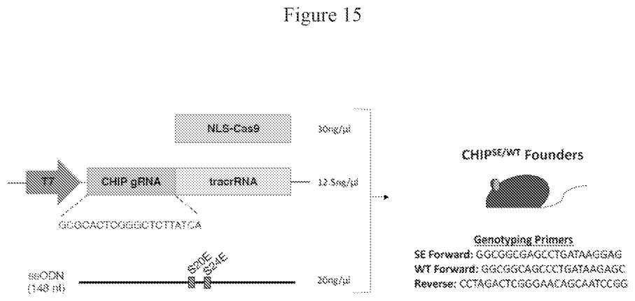

To test whether S20 phosphorylation of CHIP confers cardioprotection by enhancing PQC in vivo, global CHIPS20E knock-in (KI) mice were generated using CRISPR/Cas9 methodology (FIG. 15). Mice were born in normal Mendelian ratios and exhibited normal resting cardiac function and lifespan, as shown in Table 1.

TABLE 1

| | Mice Aged 9-12 Months | | | | |
|---|---|---|---|---|---|
| | Chip WT | n | Chip SE | n | p-value |
| Body Weight (g) | 29.8 ± 2.3 | 6 | 27.7 ± 2.5 | 7 | 0.14 |
| Heart Weight (mg) | 164.3 ± 14.9 | 6 | 147.0 ± 19.1 | 7 | 0.10 |
| Left Ventricular Weight (mg) | 119.9 ± 9.1 | 6 | 108.1 ± 13.6 | 7 | 0.10 |
| Lung Weight (mg) | 145.9 ± 17.1 | 6 | 131.8 ± 12.9 | 7 | 0.12 |
| Heart/Body Weight (mg/g) | 5.5 ± 0.3 | 6 | 5.3 ± 0.3 | 7 | 0.15 |
| Left Ventricular/Body Weight (mg/g) | 4.0 ± 0.2 | 6 | 3.9 ± 0.2 | 7 | 0.27 |
| Long/Body Weight (mg/g) | 4.9 ± 0.4 | 6 | 4.8 ± 0.2 | 7 | 0.47 |
| Heart Rate (bpm) | 701.7 ± 24.7 | 5 | 733.1 ± 59.6 | 5 | 0.31 |
| Ejection Fraction (%) | 66.9 ± 3.8 | 5 | 67.0 ± 2.1 | 5 | 0.95 |

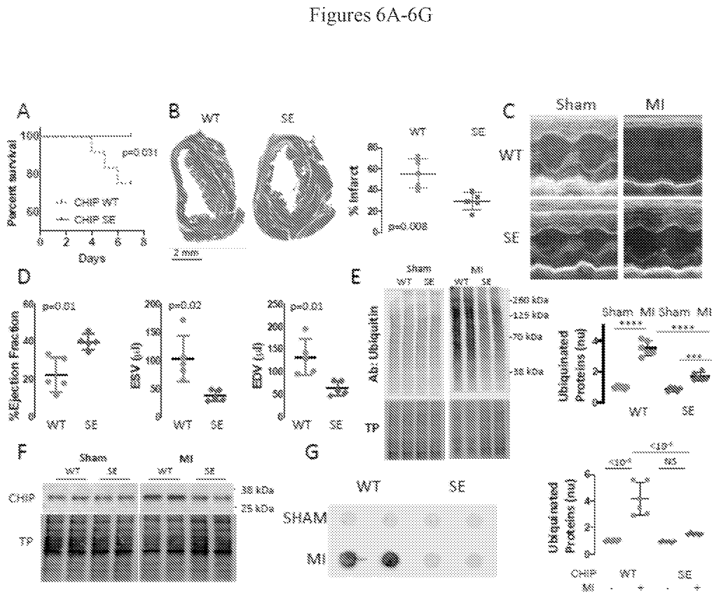

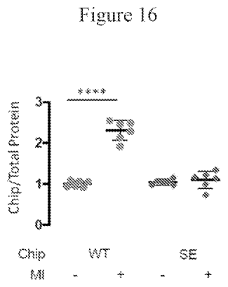

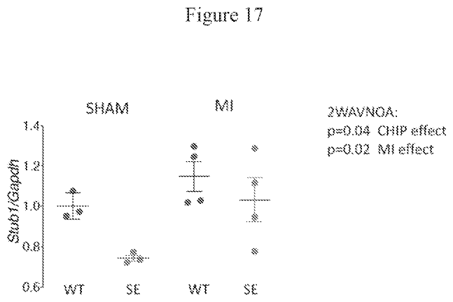

It was then tested whether expression of CHIP_S20E protects against cardiac damage/dysfunction following myocardial infarction, focusing on males as they displayed more severe post-MI disease. Compared to littermate controls, CHIP_S20E (both homozygotes and heterozygotes) subjected to MI exhibited similar lower mortality (FIG. 6A), reduced infarction size (FIG. 6B), and improved heart function (FIGS. 6C, 6D). There was also less accumulation of ubiquitinated proteins (FIG. 6E). Whereas CHIP protein level rose after MI in controls, it was unaltered in KI mice (FIG. 6F, FIG. 16), perhaps reflecting 100% of the mutant CHIP being pseudo-phosphorylated. CHIP_S20E gene expression was slightly lower than WT (FIG. 17), which could reflect longer CHIP protein half-life. To test for CHIP_S20E enhanced functionality, it was determined whether the mutation influenced clearance of insoluble protein aggregates. To this end, insoluble protein aggregates were trapped by dot blot filter assay, and stained for ubiquitin. CHIP_S20E hearts had a marked clearance of ubiquitinated proteins after MI (FIG. 6G).

Thus, the results of this example demonstrate that expression of CHIP-S20E at endogenous levels is cardioprotective after MI in vivo and improves proteostasis.

### Example 7

This example demonstrates that cardiomyocytes expressing CHIP SE exhibit reduced amyloidogenesis as compared to CHIP SA myocytes.

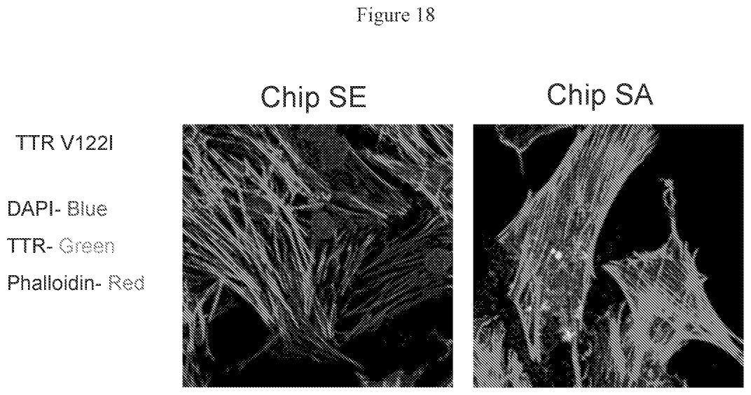

Neonatal rat cardiomyocytes (NRCMs) were cultured in mutant transthyretin (TTR) valine 122 to isoleucine (V122I)-containing medium (5 μM) and transfected with a Chip SE (serine 20 phospho-mimetic) or SA (serine 19 phospho-null) adenovirus and cultured for 14 days. Cardiomyocytes were stained with phalloidin (red), TTR (green), and nuclei with DAPI (blue). Confocal microscopy revealed increased TTR positive aggregates in Chip SA NRCMs but not in Chip SE NRCMs, as shown in FIG. 18.

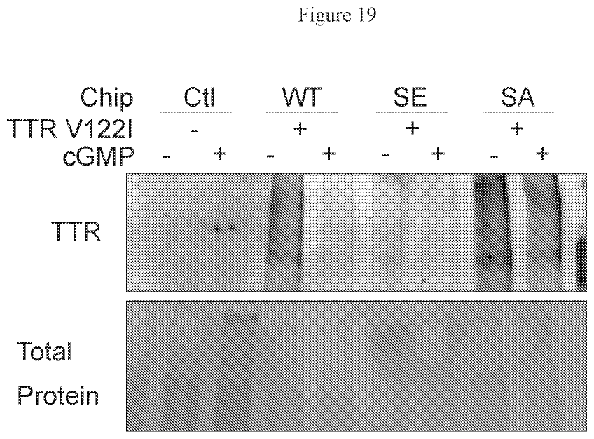

Human induced pluripotent stem cell derived cardiomyocytes (hiPSC-CMs) were infected with Ad-Chip WT, SE, or SA (10 MOI), in the presence or absence of protein kinase G (PKG) stimulation via cGMP (100 mM) and exposed to media containing TTR-V122I (5 μM) or control for 14 days. Cells were lysed and TTR abundance was determined by western blotting for TTR with a total protein stain used to show equal loading. As shown in FIG. 19, TTR accumulated in the Chip WT hiPSC-CMs, even more so with Chip SA expression, but was cleared by Chip SE expression. Stimulating PKG was able to reduce TTR accumulation in Chip WT cells but not in Chip SA cells since the serine 19 phosphosite was mutated and silenced.

### Example 8

This example demonstrates that Chip SE removal of transthyretin aggregates is dependent on Lamp2 expression.

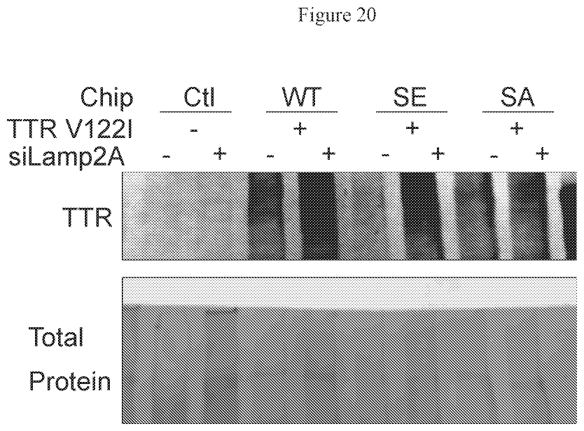

Human induced pluripotent stem cell derived cardiomyocytes (hiPSC-CMs) were infected with Ad-Chip WT, SE, or SA (10 MOI), in the presence or absence of Lamp2A knockdown via siRNA (siLamp2A, 100 μM) and exposed to media containing TTR-V122I (5 μM) or control for 14 days. Cells were lysed and TTR abundance was determined by western blotting for TTR with a total protein stain used to show equal loading. Knocking down Lamp2A removes the method of protein degradation known as chaperone mediated autophagy (CMA). As shown in FIG. 20, TTR accumulated in the Chip WT hiPSC-CMs, even more so with Chip SA expression, but was cleared by Chip SE expression. Knocking down Lamp2A exacerbated the accumulation of TTR in the Chip WT and SA cells and removed the protective effect of Chip SE. These results suggest that Chip degrades TTR via chaperone mediated autophagy.

### Example 9

This example demonstrates that Chip SE expression dose-dependently reduces TTR accumulation in cardiac fibroblasts.

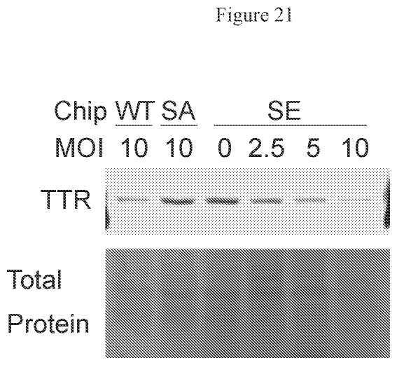

Fibroblasts were isolated from neonatal rat hearts and infected with Ad-Chip WT or SA (10 MOI) or SE (0, 2.5, 5, or 10 MOI) and exposed to media containing TTR-V122I (5 μM) for 14 days. Cells were lysed and TTR abundance was determined by western blotting for TTR with a total protein stain used to show equal loading. As shown in FIG. 21, TTR accumulated in the Chip WT fibroblasts, even more so with Chip SA expression, but was cleared by Chip SE expression in a dose-dependent manner.

### REFERENCES

All references, including publications, patent applications, and patents, cited herein are hereby incorporated by reference to the same extent as if each reference were individually and specifically indicated to be incorporated by reference and were set forth in its entirety herein.
1. Ciechanover, A. The unravelling of the ubiquitin system. Nature reviews. Molecular cell biology 16, 322-324, doi:10.1038/nrm3982 (2015).
2. Sandri, M. & Robbins, J. Proteotoxicity: an underappreciated pathology in cardiac disease. J Mol Cell Cardiol 71, 3-10, doi:10.1016/j.yjmcc.2013.12.015 (2014).
3. Hu, C. et al. Inadequate ubiquitination-proteasome coupling contributes to myocardial ischemia-reperfusion injury. J Clin Invest 128, 5294-5306, doi:10.1172/jci98287 (2018).
4. Zheng, Q. et al. Dysregulation of Ubiquitin-Proteasome System in Neurodegenerative Diseases. Frontiers in aging neuroscience 8, 303, doi:10.3389/fnagi.2016.00303 (2016).
5. Ballinger, C. A. et al. Identification of CHIP, a novel tetratricopeptide repeat-containing protein that interacts with heat shock proteins and negatively regulates chaperone functions. Mol Cell Biol 19, 4535-4545 (1999).
6. Ranek, M. J., Stachowski, M. J., Kirk, J. A. & Willis, M. S. The role of heat shock proteins and co-chaperones in heart failure. Philos Trans R Soc Lond B Biol Sci 373, doi:10.1098/rstb.2016.0530 (2018).
7. Stankiewicz, M., Nikolay, R., Rybin, V. & Mayer, M. P. CHIP participates in protein triage decisions by preferentially ubiquitinating Hsp70-bound substrates. FEBS J 277, 3353-3367, doi:10.1111/j.1742-4658.2010.07737.x (2010).
8. Narayan, V. et al. Protein-Protein Interactions Modulate the Docking-Dependent E3-Ubiquitin Ligase Activity of Carboxy-Terminus of Hsc70-Interacting Protein (CHIP). Molecular & cellular proteomics: MCP 14, 2973-2987, doi:10.1074/mcp.M115.051169 (2015).
9. Sha, Y., Rao, L., Settembre, C., Ballabio, A. & Eissa, N. T. STUB1 regulates TFEBinduced autophagy-lysosome pathway. Embo j 36, 2544-2552, doi:10.15252/embj.201796699 (2017).
10. Schisler, J. C. et al. CHIP protects against cardiac pressure overload through regulation of AMPK. J Clin Invest 123, 3588-3599, doi:10.1172fjci69080 (2013).
11. Zhang, C., Xu, Z., He, X. R., Michael, L. H. & Patterson, C. CHIP, a cochaperone/ubiquitin ligase that regulates protein quality control, is required for maximal cardioprotection after myocardial infarction in mice. Am J Physiol Heart Circ Physiol 288, H2836-2842, doi:10.1152/ajpheart.01122.2004 (2005).
12. Tawo, R. et al. The Ubiquitin Ligase CHIP Integrates Proteostasis and Aging by Regulation of Insulin Receptor Turnover. Cell 169, 470-482.e413, doi:10.1016/j.cell.2017.04.003 (2017).
13. Shi, C. H. et al. Disrupted structure and aberrant function of CHIP mediates the loss of motor and cognitive function in preclinical models of SCAR16. PLoS genetics 14, e1007664, doi:10.1371fjournal.pgen.1007664 (2018).
14. Pratt, W. B., Gestwicki, J. E., Osawa, Y. & Lieberman, A. P. Targeting Hsp90/Hsp70-based protein quality control for treatment of adult onset neurodegenerative diseases. Annual review of pharmacology and toxicology 55, 353-371, doi:10.1146/annurevpharmtox-010814-124332 (2015).
15. Kajiro, M. et al. The ubiquitin ligase CHIP acts as an upstream regulator of oncogenic pathways. Nat Cell Biol 11, 312-319, doi:10.1038/ncb1839 (2009).
16. Singh, A. K. & Pati, U. CHIP stabilizes amyloid precursor protein via proteasomal degradation and p53-mediated trans-repression of beta-secretase. Aging cell 14, 595-604, doi:10.1111/acel.12335 (2015).
17. Xu, C. W., Zhang, T. P., Wang, H. X., Yang, H. & Li, H. H. CHIP enhances angiogenesis and restores cardiac function after infarction in transgenic mice. Cellular physiology and biochemistry: international journal of experimental cellular physiology, biochemistry, and pharmacology 31, 199-208, doi:10.1159/000343361 (2013).
18. Cabral Miranda, F. et al. CHIP, a carboxy terminus HSP-70 interacting protein, prevents cell death induced by endoplasmic reticulum stress in the central nervous system. Frontiers in cellular neuroscience 8, 438, doi:10.3389/fncel.2014.00438 (2014).
19. Scaglione, K. M. et al. Ube2w and ataxin-3 coordinately regulate the ubiquitin ligase CHIP. Mol Cell 43, 599-612, doi:10.1016/j.molcel.2011.05.036 (2011).
20. Beausoleil, S. A., Villen, J., Gerber, S. A., Rush, J. & Gygi, S. P. A probability-based approach for high-throughput protein phosphorylation analysis and site localization. Nat Biotechnol 24, 1285-1292, doi:10.1038/nbt1240 (2006).
21. Olsen, J. V. et al. Global, in vivo, and site-specific phosphorylation dynamics in signaling networks. Cell 127, 635-648, doi:10.1016/j.cell.2006.09.026 (2006).
22. Ranek, M. J., Terpstra, E. J., Li, J., Kass, D. A. & Wang, X. Protein kinase g positively regulates proteasome-mediated degradation of misfolded proteins. Circulation 128, 365-376, doi:10.1161/CIRCULATIONAHA.113.001971 (2013).
23. Ranek, M. J., Kost, C. K., Jr., Hu, C., Martin, D. S. & Wang, X. Muscarinic 2 receptors modulate cardiac proteasome function in a protein kinase G-dependent manner. J Mol Cell Cardiol 69, 43-51, doi:10.1016/j.yjmcc.2014.01.017 (2014).
24. Zhang, M. et al. Myocardial remodeling is controlled by myocyte-targeted gene regulation of phosphodiesterase type 5. J Am Coll Cardiol 56, 2021-2030 (2010).
25. Rainer, P. P. et al. Cardiomyocyte-specific transforming growth factor beta suppression blocks neutrophil infiltration, augments multiple cytoprotective cascades, and reduces early mortality after myocardial infarction. Circulation research 114, 1246-1257, doi:10.1161/CIRCRESAHA.114.302653 (2014).
26. Lee, D. I. et al. Phosphodiesterase 9A controls nitric-oxide-independent cGMP and hypertrophic heart disease. Nature 519, 472-476, doi:10.1038/nature14332 (2015).
27. Ehe, B. K. et al. Data on peptides identified by mass spectrometry analysis of in vitro DYRK 1A-mediated phosphorylation sites on GLI1. Data Brief 15, 577-583, doi:10.1016/j.dib.2017.09.057 (2017).
28. Ranek, M. J. et al. PKG1-modified TSC2 regulates mTORC1 activity to counter adverse cardiac stress. Nature 566, 264-269, doi:10.1038/s41586-019-0895-y (2019).
29. Zhang, H. et al. A bipartite interaction between Hsp70 and CHIP regulates ubiquitination of chaperoned client proteins. Structure (London, England: 1993) 23, 472-482, doi:10.1016/j.str.2015.01.003 (2015).
30. Naito, A. T. et al. Promotion of CHIP-mediated p53 degradation protects the heart from ischemic injury. Circulation research 106, 1692-1702, doi:10.1161/CIRCRESAHA.109.214346 (2010).
31. Brehm, A. & Kruger, E. Dysfunction in protein clearance by the proteasome: impact on autoinflammatory diseases. Seminars in immunopathology 37, 323-333, doi:10.1007/s00281-015-0486-4 (2015).
32. Branicky, R. & Hekimi, S. Proteostasis or Aging: Let the CHIPs Fall Where They May. Developmental cell 41, 126-128, doi:10.1016/j.devcel.2017.04.006 (2017).
33. Cao, Z. et al. CHIP: A new modulator of human malignant disorders. Oncotarget 7, 29864-29874, doi:10.18632/oncotarget.8219 (2016).
34. Xiao, M., Yan, M., Zhang, J., Xu, Q. & Chen, W. Carboxy-terminus Hsc70 interacting protein exerts a tumor inhibition function in head and neck cancer. Oncol Rep 38, 1629-1636, doi:10.3892/or.2017.5827 (2017).
35. Kim, C. et al. Phosphorylation of CHIP at Ser20 by Cdk5 promotes tAIF-mediated neuronal death. Cell Death Differ, doi:10.1038/cdd.2015.103 (2015).
36. Li, J. M., Poolman, R. A. & Brooks, G. Role of Gi phase cyclins and cyclin-dependent kinases during cardiomyocyte hypertrophic growth in rats. Am J Physiol 275, H814-822 (1998).
37. Koitabashi, N. et al. Cyclic GMP/PKG-dependent inhibition of TRPC6 channel activity and expression negatively regulates cardiomyocyte NFAT activation Novel mechanism of cardiac stress modulation by PDE5 inhibition. J Mol Cell Cardiol 48, 713-724 (2010).
38. Takimoto, E. et al. Regulator of G protein signaling 2 mediates cardiac compensation to pressure overload and antihypertrophic effects of PDE5 inhibition in mice. J Clin Invest 119, 408-420 (2009).
39. Oeing, C. U. et al. PKG1alpha Cysteine-42 Redox State Controls mTORC1 Activation in Pathological Cardiac Hypertrophy. Circ Res, doi:10.1161/CIRCRESAHA.119.315714 (2020).
40. VerPlank, J. J. S., Tyrkalska, S. D., Fleming, A., Rubinsztein, D. C. & Goldberg, A. L. cGMP via PKG activates 26S proteasomes and enhances degradation of proteins, including ones that cause neurodegenerative diseases. Proc Natl Acad Sci USA, doi:10.1073/pnas.2003277117 (2020).
41. Kaushik, S. & Cuervo, A. M. The coming of age of chaperone-mediated autophagy. Nature reviews. Molecular cell biology 19, 365-381, doi:10.1038/s41580-018-0001-6 (2018).
42. Arndt, V. et al. Chaperone-assisted selective autophagy is essential for muscle maintenance. Current biology: CB 20, 143-148, doi:10.1016fj.cub.2009.11.022 (2010).
43. Dai, Q. et al. CHIP activates HSF1 and confers protection against apoptosis and cellular stress. The EMBO journal 22, 5446-5458, doi:10.1093/emboj/cdg529 (2003).
44. Dikshit, P. & Jana, N. R. The co-chaperone CHIP is induced in various stresses and confers protection to cells. Biochem Biophys Res Commun 357, 761-765 (2007).
45. Nakamura, T. et al. Prevention of PKG1alpha oxidation augments cardioprotection in the stressed heart. The Journal of clinical investigation 125, 2468-2472, doi:10.1172/JC180275 (2015).
46. Breitenstein, S., Roessig, L., Sandner, P. & Lewis, K. S. Novel sGC Stimulators and sGC Activators for the Treatment of Heart Failure. Handbook of experimental pharmacology 243, 225-247, doi:10.1007/164_2016_100 (2017).
47. McMurray, J. J. et al. Angiotensin-neprilysin inhibition versus enalapril in heart failure. N EnglJMed 371, 993-1004, doi:10.1056/NEJMoa1409077 (2014).

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inhibiting toxic protein accumulation in a cell, which method comprises contacting a cell with a nucleic acid sequence encoding a carboxy terminus of heat shock cognate 70 interacting protein (CHIP), wherein the CHIP protein comprises at least one phospho-mimetic amino acid substitution at position 19 of SEQ ID NO: 1 or at position 20 of SEQ ID NO: 2, whereby the nucleic acid sequence is expressed in the cell and toxic protein accumulation is inhibited.

2. The method of claim 1, wherein the at least one phospho-mimetic amino acid substitution is (a) a substitution of the serine residue at position 19 of SEQ ID NO: 1 for glutamic acid (S19E) or aspartic acid (S19D) or (b) a substitution of the serine residue at position 20 of SEQ ID NO: 2 for glutamic acid residue (S20E) or aspartic acid (S20D).

3. The method of claim 1 or claim 2, wherein the cell is in vitro.

4. The method of claim 1 or claim 2, wherein the cell is in vivo.

5. The method of claim 4, wherein the cell is in a human and the nucleic acid sequence is present in a composition comprising a pharmaceutically acceptable carrier.

6. The method of claim 5, wherein the human is suffering from cardiac dysfunction or neurological dysfunction.

7. The method of claim 6, wherein the cardiac dysfunction is myocardial ischemia-reperfusion injury or myocardial infarction.

8. The method of claim 6, wherein the cardiac dysfunction is the result of amyloid accumulation in the myocardium.

9. The method of claim 8, wherein the cardiac dysfunction is transthyretin cardiac amyloidosis (ATTR-CA).

10. The method of claim 6, wherein the neurological dysfunction is Alzheimer's disease, Huntington's disease, Parkinson's disease, prion disease, or amyotrophic lateral sclerosis (ALS).

11. The method of claim 5, wherein the human is suffering from a proteinopathy.

12. The method of claim 11, wherein the proteinopathy is selected from cystic fibrosis transmembrane regulator (CFTR) opathies, laminopathies, inflammasomeopathies, adiposopathy, telomeropathies, Fanconi anemia bloom syndrome (FANC-BLM) opathies, lysosomopathies, phosphatopathies, phosphotase and tensin homolog deleted on chromosome 10 (PTEN) opathies, caspase recruitment domain family member 11 (CARD11 or CARMA1)—B cell CLL/lymphoma 10 (BCL10)—MALT1 paracaspase (MALT1) (CBM) opathies, phosphodiesterase (PDE) opathies, BCID cargo adapter 2 (BICD2) opathies, Behcet's disease, axonopathy, dysferopathy, synaptopathy, clippers and myelin oligodendrocyte glycoprotein (MOG) opathy, RAS/mitogen-activated protein kinase (RAS) opathies, protein kinase R-like ER kinase (PERK) opathies, Bcl-2-associated athanogene (BAG) opathies (e.g., Bcl-2-associated athanogene-1 gene(BAG1) opathy or Bcl-2-associated athanogene-3 gene (BAG3) opathy), BRG1/BRM-associated factor (BAF) opathies/SWI/SNF-related intellectual disability disorder (SSRIDD), four and a half LIM domain 1 (FHL1) opathies, apolipoprotein E (APOE) opathies, tauopathies, Abetaopathies (also referred to as “amyloid beta-opathies”), Huntington's disease (e.g., Huntingtonoapthy), alpha-synucleinopathies (e.g., synucleinopathies), multiple sclerosis, transactive response DNA-binding protein 43 (TDP-43) opathies, fused-in-sarcoma (FUS) opathies, superoxide dismutase 1 (SOD1) opathies, amyotrophic lateral sclerosis (ALS), microtubuleopathies, heat shock protein (HSP) opathies, chaperoneopathies, Paget's disease, osteoprotegerinopathies, glycogen synthase kinase 3 beta (GSK3beta)opathies, oligodendrogliopathies, fronto-temporal lobar degeneration (FTLD), British and Danish dementias, cerebral autosomal dominant arteriopathy with subcortical infarcts and leukoencephalopathy (CADASIL), neurogenic locus notch homolog protein 3 (NOTCH3) opathies, prion disease, Creutzfeldt-Jakob disease, Pick's disease, traumatic brain injury (TBI), hypertrophic cardiomyopathy, cardiac arrhythmias, channelopathies, Noonan syndrome, sarcomeropathies, and myofibrillar myopathy.

13. The method of any one of claims 1-12, wherein the nucleic acid sequence is present in a vector.

14. The method of claim 13, wherein the vector is a viral vector.

15. A method of inhibiting toxic protein accumulation in a cell, which method comprises increasing or inducing in the cell phosphorylation of a serine residue at (i) position 19 of a human carboxy terminus of heat shock cognate 70 interacting protein (CHIP) comprising the amino acid sequence of SEQ ID NO: 1 or at (ii) position 20 of a mouse CHIP protein comprising the amino acid sequence of SEQ ID NO: 2.

16. The method of claim 15, wherein the cell is in vitro.

17. The method of claim 15, wherein the cell is in vivo.

18. The method of claim 17, wherein the cell is in a human who is suffering from cardiac dysfunction or neurological dysfunction.

19. The method of claim 18, wherein the cardiac dysfunction is myocardial ischemia-reperfusion injury or myocardial infarction.

20. The method of claim 18, wherein the cardiac dysfunction is the result of amyloid accumulation in the myocardium.

21. The method of claim 20, wherein the cardiac dysfunction is transthyretin cardiac amyloidosis (ATTR-CA).

22. The method of claim 17, wherein the cell is in a human who is suffering from a proteinopathy.

23. The method of claim 22, wherein the proteinopathy is selected from cystic fibrosis transmembrane regulator (CFTR) opathies, laminopathies, inflammasomeopathies, adiposopathy, telomeropathies, Fanconi anemia bloom syndrome (FANC-BLM) opathies, lysosomopathies, phosphatopathies, phosphotase and tensin homolog deleted on chromosome 10 (PTEN) opathies, caspase recruitment domain family member 11 (CARD11 or CARMA1)—B cell CLL/lymphoma 10 (BCL10)—MALT1 paracaspase (MALT1) (CBM) opathies, phosphodiesterase (PDE) opathies, BCID cargo adapter 2 (BICD2) opathies, Behcet's disease, axonopathy, dysferopathy, synaptopathy, clippers and myelin oligodendrocyte glycoprotein (MOG) opathy, RAS/mitogen-activated protein kinase (RAS) opathies, protein kinase R-like ER kinase (PERK) opathies, Bcl-2-associated athanogene (BAG) opathies (e.g., Bcl-2-associated athanogene-1 gene(BAG1) opathy or Bcl-2-associated athanogene-3 gene (BAG3) opathy), BRG1/BRM-associated factor (BAF) opathies/SWI/SNF-related intellectual disability disorder (SSRIDD), four and a half LIM domain 1 (FHL1) opathies, apolipoprotein E (APOE) opathies, tauopathies, Abetaopathies (also referred to as “amyloid beta-opathies”), Huntington's disease (e.g., Huntingtonoapthy), alpha-synucleinopathies (e.g., synucleinopathies), multiple sclerosis, transactive response DNA-binding protein 43 (TDP-43) opathies, fused-in-sarcoma (FUS) opathies, superoxide dismutase 1 (SOD1) opathies, amyotrophic lateral sclerosis (ALS), microtubuleopathies, heat shock protein (HSP) opathies, chaperoneopathies, Paget's disease, osteoprotegerinopathies, glycogen synthase kinase 3 beta (GSK3beta)opathies, oligodendrogliopathies, fronto-temporal lobar degeneration (FTLD), British and Danish dementias, cerebral autosomal dominant arteriopathy with subcortical infarcts and leukoencephalopathy (CADASIL), neurogenic locus notch homolog protein 3 (NOTCH3) opathies, prion disease, Creutzfeldt-Jakob disease, Pick's disease, traumatic brain injury (TBI), hypertrophic cardiomyopathy, cardiac arrhythmias, channelopathies, Noonan syndrome, sarcomeropathies, and myofibrillar myopathy.

24. The method of claim 18, wherein the neurological dysfunction is Alzheimer's disease, Huntington's disease, Parkinson's disease, prion disease, or amyotrophic lateral sclerosis (ALS).

25. The method of any one of claims 15-25, wherein increasing or inducing phosphorylation of a serine residue comprises increasing or inducing the activity of protein kinase G (PKG) in the cell.

26. The method of claim 25, wherein increasing or inducing the activity of PKG in the cell comprises contacting the cell with an agent that stimulates PKG activity.

27. The method of claim 26, wherein the agent is selected from nitric oxide donors, organic nitrates, soluble guanylate cyclase stimulators, PDE5 inhibitors that enhance NO-dependent cGMP synthesis, natriuretic peptides (NP), neprilysin inhibitors, and PDE9 inhibitors that increase NP-dependent cGMP stimulation.

28. A method of treating a disease caused by proteotoxicity in a cell, which comprises increasing or inducing the activity of carboxy terminus of heat shock cognate 70 interacting protein (CHIP) in a subject in need thereof.

29. The method of claim 28, wherein increasing or inducing the activity of CHIP comprises administering to the subject a composition comprising a nucleic acid sequence encoding a human CHIP protein and a pharmaceutically acceptable carrier, wherein the human CHIP protein comprises at least one phospho-mimetic amino acid substitution at position 19 of SEQ ID NO: 1, whereby the nucleic acid sequence is expressed in subject and the disease is treated.

30. The method of 29, wherein the at least one phospho-mimetic amino acid substitution is a substitution of the serine residue at position 19 of SEQ ID NO: 1 for glutamic acid (SI9E) or aspartic acid (S19D).

31. The method of claim 28, wherein increasing or inducing the activity of CHIP comprises increasing or inducing the activity of protein kinase G (PKG) in the cells of the subject.

32. The method of claim 31, wherein increasing or inducing the activity of PKG in the cell comprises contacting the cell with an agent that stimulates PKG activity.

33. The method of claim 32, wherein the agent is selected from nitric oxide donors, organic nitrates, soluble guanylate cyclase stimulators, PDE5 inhibitors that enhance NO-dependent cGMP synthesis, natriuretic peptides (NP), neprilysin inhibitors, and PDE9 inhibitors that increase NP-dependent cGMP stimulation.

34. The method of any one of claims 28-33, wherein the disease is a cardiac disease or a neurological disease.

35. The method of claim 34, wherein the cardiac disease is myocardial ischemia-reperfusion injury or myocardial infarction.

36. The method of claim 34, wherein the cardiac disease is the result of amyloid accumulation in the myocardium.

37. The method of claim 36, wherein the cardiac disease is transthyretin cardiac amyloidosis (ATTR-CA).

38. The method of claim 34, wherein the neurological disease is Alzheimer's disease, Huntington's disease, Parkinson's disease, prion disease, or amyotrophic lateral sclerosis (ALS).

39. The method of any one of claims 28-33, wherein the disease is a proteinopathy.

40. The method of claim 39, wherein the proteinopathy is selected from cystic fibrosis transmembrane regulator (CFTR) opathies, laminopathies, inflammasomeopathies, adiposopathy, telomeropathies, Fanconi anemia bloom syndrome (FANC-BLM) opathies, lysosomopathies, phosphatopathies, phosphotase and tensin homolog deleted on chromosome 10 (PTEN) opathies, caspase recruitment domain family member 11 (CARD11 or CARMA1)—B cell CLL/lymphoma 10 (BCL10)—MALT1 paracaspase (MALT1) (CBM) opathies, phosphodiesterase (PDE) opathies, BCID cargo adapter 2 (BICD2) opathies, Behcet's disease, axonopathy, dysferopathy, synaptopathy, clippers and myelin oligodendrocyte glycoprotein (MOG) opathy, RAS/mitogen-activated protein kinase (RAS) opathies, protein kinase R-like ER kinase (PERK) opathies, Bcl-2-associated athanogene (BAG) opathies (e.g., Bcl-2-associated athanogene-1 gene(BAG1) opathy or Bcl-2-associated athanogene-3 gene (BAG3) opathy), BRG1/BRM-associated factor (BAF) opathies/SWI/SNF-related intellectual disability disorder (SSRIDD), four and a half LIM domain 1 (FHL1) opathies, apolipoprotein E (APOE) opathies, tauopathies, Abetaopathies (also referred to as “amyloid beta-opathies”), Huntington's disease (e.g., Huntingtonoapthy), alpha-synucleinopathies (e.g., synucleinopathies), multiple sclerosis, transactive response DNA-binding protein 43 (TDP-43) opathies, fused-in-sarcoma (FUS) opathies, superoxide dismutase 1 (SOD1) opathies, amyotrophic lateral sclerosis (ALS), microtubuleopathies, heat shock protein (HSP) opathies, chaperoneopathies, Paget's disease, osteoprotegerinopathies, glycogen synthase kinase 3 beta (GSK3beta)opathies, oligodendrogliopathies, fronto-temporal lobar degeneration (FTLD), British and Danish dementias, cerebral autosomal dominant arteriopathy with subcortical infarcts and leukoencephalopathy (CADASIL), neurogenic locus notch homolog protein 3 (NOTCH3) opathies, prion disease, Creutzfeldt-Jakob disease, Pick's disease, traumatic brain injury (TBI), hypertrophic cardiomyopathy, cardiac arrhythmias, channelopathies, Noonan syndrome, sarcomeropathies, and myofibrillar myopathy.

41. Use of a nucleic acid sequence encoding a carboxy terminus of heat shock cognate 70 interacting protein (CHIP) for inhibiting toxic protein accumulation in a cell, wherein the CHIP protein comprises at least one phospho-mimetic amino acid substitution at position 19 of SEQ ID NO: 1 or at position 20 of SEQ ID NO: 2.

42. Use of a nucleic acid sequence encoding a carboxy terminus of heat shock cognate 70 interacting protein (CHIP)in the treatment of a disease caused by proteotoxicity, wherein the CHIP protein comprises at least one phospho-mimetic amino acid substitution at position 19 of SEQ ID NO: 1 or at position 20 of SEQ ID NO: 2.

43. The use of claim 41 or claim 42, wherein the at least one phospho-mimetic amino acid substitution is (a) a substitution of the serine residue at position 19 of SEQ ID NO: 1 for glutamic acid (S19E) or aspartic acid (S19D) or (b) a substitution of the serine residue at position 20 of SEQ ID NO: 2 for glutamic acid residue (S20E) or aspartic acid (S20D).
